(19) United States
(12) Patent Application Publication
(10) Pub. No.: US 2008/0131525 A1
(43) Pub. Date: Jun. 5, 2008

(54) Therapeutic Nutrient Compositions Or Combinations And Methods Of Their Use

(76) Inventor: Daren Heyland, Kingston (CA)

Correspondence Address: ARENT FOX LLP, 1050 CONNECTICUT AVENUE, N.W., SUITE 400, WASHINGTON, DC, 20036, US

(21) Appl. No.: 11/792,587

(22) PCT Filed: Dec. 21, 2005
(86) PCT No.: PCT/CA05/01944

§ 371 Date: Jun. 8, 2007

Related U.S. Application Data
(60) Provisional application No. 60/637,657, filed on Dec. 21, 2004.

(51) Int. Cl. A61K 38/00; A61K 33/04; A61K 33/30; A61P 39/06

(52) U.S. Cl. 424/641; 514/2; 424/702

(57) ABSTRACT

The invention may be summarized as follows. A combination to be parenterally delivered to a critically ill patient or for the purpose of improving mitochondrial function. The combination comprises a glutamine precursor molecule and an antioxidant in sufficient concentrations to be clinically effective. The combination may be prepared in the absence of lipids or carbohydrates. The combination may be prepared in small volumes to benefit volume restricted patients. A composition, or a unit dosage form comprising the combination and methods of administering the combination, composition or unit dosage form are also provided.

### FIELD OF INVENTION

The present invention relates to a nutrient composition that can be used for treatment of a critically ill patient, or for improving mitochondrial function. More particularly, the present invention relates to a use of a composition comprising a high concentration of an amino acid, an antioxidant, or a combination thereof for treatment of a critically ill patient, or for improving mitochondrial function.

### BACKGROUND OF THE INVENTION

The relationship between nutrient deficiency and altered immune status has been recognized for years. Morever, certain critical care conditions can further exacerbate nutrient deficiencies predisposing patients to impaired immune function and higher risk of developing infectious complications, organ dysfunction, and death. Consequently, over the last few decades numerous experimental studies have explored the immune-modulating properties of nutrients such as glutamine, arginine, omega-3 fatty acids, and others. Several nutrition formulas supplemented with one or more of these nutrients have been developed and are currently available. “Immunonutrition”, “immune-enhancing diets”, and other terms have been used to describe these products. Unfortunately, these products have been developed without a sound scientific understanding of what effect these nutrients have on clinically important outcomes in the critical care setting.

A treatment benefit from various substrates or nutrients will vary depending on the underlying pathophysiology of the host and whether the substrate influences cellular immune function and/or the synthesis of inflammatory mediators and/or the generation of reactive oxygen species (ROS) and/or mitochondrial function. A minimum level of key nutrients (glutamine, arginine, and omega-3 fatty acids) is required for immunocompetence. However, particularly in the case of arginine which produces excessive nitric oxide (NO) production and omega-3 fatty acids which produces eicosanoid synthesis, excessive amounts of these nutrients may have immunodepressant effects and may be associated with worse clinical outcomes.

Given this heterogenous and variable treatment response, one cannot look at the clinical trials of immunonutrition in surgical patients (or in patients with AIDS, obesity, etc.) and generalize the results to critically ill patients. Generally speaking, elective surgical patients experience minimal activation of cytokines and some degree of suppression of the cellular defense function following surgical stress putting them at higher risk for acquired infectious morbidity and mortality. It follows that nutrients that stimulate the cellular defense system may reduce infectious complications in the elective surgical patient. In contrast, the associated changes to the systemic inflammatory response accompanying critical illness are far more intense, complex, variable, and less well defined.

Recommended parenteral nutrition intakes or standard doses of micronutrients are based on requirements and metabolism in healthy subjects and have little meaning in critically ill patients. At high doses, vitamin C, vitamin E and selenium have been shown to have some pro-oxidant properties (Abuja P M: When might an antioxidant become a prooxidant? Acta Anaesthesiol Scand 1998; 42(Suppl 112):229-230; Spallholz J E: The negative effects of excessive amounts of naturally occurring selenium. The Selenium-Tellurium Development Association 1998; February). Therefore, more may not necessarily result in better outcomes. Further research is needed to determine desired doses of micronutrients that can have a beneficial effect on clinical outcomes, particularly when given in combination with glutamine. In addition, there are several difficulties in providing a high amount of free glutamine to critically ill patients due to problems with limited solubility, and stability, especially in patients with volume-restricted conditions.

Mitochondrial dysfunction can be a problem in critically ill patients due to a number of factors including, without limitation, damage from reactive oxygen species (ROS) or toxic side effect of therapeutic compounds. Other patient groups, such as cancer patients, may also experience mitochondrial dysfunction as a side effect of an oncology treatment protocol. Other patient groups, such as AIDS/HIV patients, may also experience mitochondrial dysfunction as a side effect of an antiviral treatment protocol. Still other patient groups may be genetically predisposed to mitochondrial dysfunction. Accordingly, a method for improving mitochondrial function may benefit critically ill patients as well as other patients suffering from mitochondrial dysfunction.

Thus, there is a need to determine key nutrients, and their routes of delivery that may provide beneficial outcomes in treatment of the critically ill or for improving mitochondrial function.

### SUMMARY OF THE INVENTION

The present invention relates to a nutrient composition that can be used for treatment of a critically ill patient, or for improving mitochondrial function. More particularly, the present invention relates to a use of a composition comprising a high concentration of an amino acid, an antioxidant, or a combination thereof for treatment of a critically ill patient, or for improving mitochondrial function.

It is an object of the invention to provide an improved therapeutic nutrient composition, therapeutic nutrient combination, or method of their use.

According to an aspect of the present invention there is provided a composition comprising glutamine from about 35 to about 380 grams or any range or amount therebetween per litre of solution provided as a short chain peptide and an antioxidant selected from the group consisting of selenium at a concentration from about 400 to about 10000 micrograms or any range or amount therebetween per litre, vitamin C at a concentration from about 1000 to about 20000 milligrams or any range or amount therebetween per litre, zinc at a concentration from about 20 to about 800 milligrams or any range or amount therebetween per litre, vitamin E at a concentration from about 500 to about 12000 milligrams or any range or amount therebetween per litre, beta-carotene at a concentration from about 20 to about 4000 milligrams or any range or amount therebetween per litre, and combinations thereof.

The above composition may be delivered parenterally for treatment of a critically ill patient, or for improving mitochondrial function.

According to an aspect of the present invention there is provided a unit dosage form of about 50 to about 1000 millilitres total volume, the unit dosage form comprising glutamine from about 35 to about 380 grams or any range or amount therebetween per litre of solution provided as a short chain peptide, and an antioxidant selected from the group consisting of selenium at a concentration from about 400 to about 10000 micrograms or any range or amount therebetween per litre, vitamin C at a concentration from about 1000 to about 20000 milligrams or any range or amount therebetween per litre, zinc at a concentration from about 20 to about 800 milligrams or any range or amount therebetween per litre, vitamin E at a concentration from about 500 to about 12000 milligrams or any range or amount therebetween per litre, beta-carotene at a concentration from about 20 to about 4000 milligrams or any range or amount therebetween per litre, and combinations thereof.

The above unit dosage form may be delivered parenterally for treatment of a critically ill patient, or for improving mitochondrial function.

According to an aspect of the present invention there is provided a combination comprising glutamine from about 35 to about 380 grams or any range or amount therebetween per litre of solution provided as a short chain peptide, and an antioxidant selected from the group consisting of selenium at a concentration from about 400 to about 10000 micrograms or any range or amount therebetween per litre, vitamin C at a concentration from about 1000 to about 20000 milligrams or any range or amount therebetween per litre, zinc at a concentration from about 20 to about 800 milligrams or any range or amount therebetween per litre, vitamin E at a concentration from about 500 to about 12000 milligrams or any range or amount therebetween per litre, beta-carotene at a concentration from about 20 to about 4000 milligrams or any range or amount therebetween per litre, and combinations thereof. In certain examples, the components, of the combination are delivered simultaneously, while in other examples the components are delivered at separate times. In certain examples, the components of the combination, are delivered using the same mode of administration, while in other examples the components are delivered using different modes of administration.

The above combination may be delivered parenterally for treatment of a critically ill patient, or for improving mitochondrial function.

In certain examples, the total volume of the unit dosage may be from about 50 to about 1000 millilitres, or any range or amount therebetween. For example, the total volume may be from about 200 to about 500 millilitres. As another example, the total volume may be about 1000, 900, 800, 700, 600, 500, 450, 400, 350, 300, 250, 200, 150, 100 or 50 millilitres or any volume therebetween.

In certain examples, selenium may be used at a concentration from about 400 to about 10000 micrograms or any range or amount therebetween per litre of solution. For example, selenium concentration may be from about 1000 to about 4000 micrograms per litre. As another example, selenium may be used at concentrations of about 400, 600, 800, 1000, 1200, 1400, 1600, 1800, 2000, 2200, 2400, 2600, 2800, 3000, 3200, 3400, 3600, 3800, 4000, 4500, 5000, 5500, 6000, 6500, 7000, 8000, 9000, or 10000 micrograms per litre of solution or any concentration therebetween.

In certain examples, glutamine may be used at a concentration from about 35 to about 380 grams or any range or amount therebetween per litre of solution. For example, glutamine concentration may be about 50 to about 150 grams per litre of solution. As another example, glutamine may be used at concentrations of about 35, 40, 45, 50, 55, 60, 65, 70, 75, 80, 85, 90, 95, 100, 110, 115, 120, 125, 130, 135, 140, 150, 160, 170, 180, 190, 200, 250, 300, or 350 grams per litre of solution or any concentration therebetween.

In certain examples, the compositions, combinations or unit dosage forms of the invention may be prepared in the absence of lipids or carbohydrates.

According to an aspect of the present invention there is provided a method of treating a critically ill patient comprising administering a composition or combination of the present invention, to a critically ill patient in need of such treatment. As an example, a composition of the invention is administered parenterally to a patient in a daily dose from about 0.3 g glutamine/kg body weight to about 0.9 g glutamine/kg body weight or any range or amount therebetween. As another example, a composition of the invention is administered to a patient in a daily dose from about 400 to about 2000 micrograms selenium or any range or amount therebetween.

According to an aspect of the present invention there is provided a method of improving mitochondrial function comprising administering a composition or combination of the present invention, to a patient in need of such treatment. As an example, a composition of the invention is administered parenterally to a patient in a daily dose from about 0.3 g glutamine/kg body weight to about 0.9 g glutamine/kg body weight or any range or amount therebetween. As another example, a composition of the invention is administered to a patient in a daily dose from about 400 to about 2000 micrograms selenium or any range or amount therebetween. In certain examples, the patient is suffering from cellular degeneration associated with mitochondrial dysfunction.

An advantage of the present invention is that compositions may be formulated in small volumes, and therefore may be administered to volume restricted patients.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows plots of mean daily SOFA scores (see Table 6 for summary of SOFA scoring system) for various organ systems for patients in Group 1/Control (FIG. 1A), Group 2 (FIG. 1B), Group 3 (FIG. 1C), Group 4 (FIG. 1D), and Group 5 (FIG. 1E);

FIG. 2 shows plots of total daily SOFA scores (for each patient the SOFA scores shown for each organ system is added to calculate the total SOFA score) for patients in Group 1/Control (FIG. 2A), Group 2 (FIG. 2B), Group 3 (FIG. 2C), Group 4 (FIG. 2D), and Group 5 (FIG. 2E) with the regression lines from FIGS. 2(A-E) collected in a single plot in FIG. 2F;

FIG. 3 shows plots of glutathione (GSH) content of red blood cells for patients in Group 2 (FIG. 3A), Group 3 (FIG. 3B), Group 4 (FIG. 3C), and Group 5 (FIG. 3D) with the regression lines from FIGS. 3(A-D) collected in a single plot in FIG. 3E;

FIG. 4 shows plots of plasma concentrations of thiobarbituric acid reactive substances (TBARS; an index of lipid peroxidation and a marker of oxidative stress), for patients in Group 2 (FIG. 4A), Group 3 (FIG. 4B), Group 4 (FIG. 4C), and Group 5 (FIG. 4D) with the regression lines from FIGS. 4(A-D) collected in a single plot in FIG. 4E;

FIG. 5 shows plots of the ratio of levels of mitochondrial DNA and nuclear DNA (mtDna/nDNA; an indicator of mitochondrial function), for patients in Group 2 (FIG. 5A), Group 3 (FIG. 5B), Group 4 (FIG. 5C), and Group 5 (FIG. 5D) with the regression lines from FIGS. 5(A-D) collected in a single plot in FIG. 5E;

FIG. 6 shows plots of the mtDna/nDNA ratio for individual patients that are categorized as either alive or expired with regression lines shown in a larger point size;

FIG. 7 shows plots of the mtDna/nDNA ratio for individual patients that are categorized as either Group 2 patients or Groups 3, 4, and 5 patients with regression lines shown in a larger point size;

FIG. 8 shows regression line plots for plasma concentrations of creatinine (an indicator of kidney function or renal function), for patients in Group 1/Control, Group 2, Group 3, Group 4, and Group 5;

### DETAILED DESCRIPTION

The present invention relates to a nutrient composition that can be used for treatment of a critically ill patient, or for improving mitochondrial function. More particularly, the present invention relates to a use of a composition comprising a high concentration of an amino acid, an antioxidant, or a combination thereof for treatment of a critically ill patient, or for improving mitochondrial function.

The following description is of a preferred embodiment.

Several previous-randomized trials have failed to demonstrate a treatment effect in critically ill patients when providing key nutrients enterally (Novak et al. Glutamine supplementation in serious illness: A systematic review of the evidence, Crit. Care Med. 2002; 30; 2022-29). However, given the major role of the gastrointestinal tract as a source of cytokine and leukocyte activation and reactive oxygen species formation, providing the key nutrients directly to the lumen of the gastrointestinal tract makes theoretical sense. Without wishing to be bound by theory, a reason for the lack of observed treatment effect may be that when provided enterally and combined with the enteral nutrition product, sick patients may have trouble tolerating their enteral feeds thereby limiting the intake of key nutrients. In certain examples, the present invention dissociates the provision of these key nutrients from the provision of enteral (or parenteral) nutrition by parenterally delivering high concentrations of key nutrients without requiring the presence of macronutrients that are typically included in nutritional supplements, for example, lipids or carbohydrates. However, such macronutrients may optionally be added to or used in conjunction with the compositions of the present invention in order to benefit a particular patient or patient population.

An aspect of the present invention pertains to a composition comprising glutamine from about 35 to about 380 grams or any range or amount therebetween per litre of solution provided as a short chain peptide and an antioxidant selected from the group consisting of selenium, vitamin C, zinc, vitamin E, beta-carotene, and combinations thereof. In certain examples, two or more antioxidants may be selected. In some examples, the composition can be parenterally delivered to a patient. In further examples, the compositions can be used to treat critically ill patients. Furtherstill, the compositions can be used to treat mitochondrial dysfunction or improve mitochondrial function in patients suffering from mitochondrial dysfunction. In certain examples of the present invention, the composition may be used to treat patients that are both critically ill and suffer from mitochondrial dysfunction.

In certain examples of the present invention, a composition is to be delivered parenterally to a critically ill patient or to a patient suffering from mitochondrial dysfunction comprising glutamine from about 35 to about 380 grams or any range or amount therebetween per litre of solution provided as a short chain peptide and an antioxidant selected from the group consisting of selenium at a concentration from about 400 to about 10000 micrograms or any range or amount therebetween per litre, vitamin C at a concentration from about 1000 to about 20000 milligrams or any range or amount therebetween per litre, zinc at a concentration from about 20 to about 800 milligrams or any range or amount therebetween per litre, vitamin E at a concentration from about 500 to about 12000 milligrams or any range or amount therebetween per litre, beta-carotene at a concentration from about 20 to about 4000 milligrams or any range or amount therebetween per litre, and combinations thereof. As another example, the antioxidant is selenium at a concentration of about 1000 to about 4000 micrograms per litre, and the concentration of glutamine is from about 50 to about 100 grams per litre of solution. In certain examples the composition may comprise two or more antioxidants. In certain examples, a unit dosage form comprising the composition of the present invention has a total volume of about 50 to about 1000 millilitres or any range or amount therebetween. For example, the total volume is about 1000, 900, 800, 700, 600, 500, 450, 400, 350, 300, 250, 200, 150, 100, or 50 millilitres or any volume therebetween. Furthermore, it is preferred that lipids, carbohydrates, or both lipids and carbohydrates are absent from the composition.

“Critical illness”, “critical care”, “critically ill”, or other variations pertain to a patient requiring treatment in an Intensive Care Unit (ICU) or a patient at risk of dying or developing multiple organ failure, for example, patients exhibiting evidence of multiple organ dysfunction or evidence associated with the onset of multiple organ dysfunction, as will be recognized by one of skill in the art.

Without wishing to be bound by theory, reactive oxygen species (ROS) are assumed to play a key role in the pathophysiology underlying critical illness. ROS not only lead to direct damage of cellular components but also trigger the release of cytokines that further activate the inflammatory cascades (Grimble R F. Nutritional Antioxidants and the modulation of inflammation: Theory and practice, New Horizons 1994; 2: 175-185). Free radicals can activate resident macrophages or Kupffer cells which release inflammatory cytokines (for example, TNF, IL-1, L-6, IL-18). These pro-inflammatory mediators, in turn, elicit activation and influx of inflammatory cells (monocytes and leukocytes) into tissues and organs and may directly cause mitochondrial dysfunction leading to further ischemia and tissue injury. Furthermore, the activated Kupffer cells also produce large amounts of oxygen free radicals whereby a vicious cycle of inflammation, cellular activation, and ROS generation is created.

In a very simplistic model, the host response to invading micro organisms can be divided into two arms: 1) cellular defence that includes both innate (non-specific) immunity and adaptive (specific) immunity and 2) the systemic inflammatory response. The cellular defence function includes all functions of polymorphonuclear granulocytes, macrophages and lymphocytes as well as their proliferation behaviour. By contrast the systemic inflammatory response, which is triggered by immune competent cells, works mainly at the tissue level. The systemic inflammatory reaction is characterized by effects of the mediators, free radicals, and activated immune cells on metabolism, the endothelium, platelets, and smooth muscles of the vascular and bronchial systems.

Treatment effect of various substrates or nutrients will vary depending on the underlying pathophysiology of a patient and whether the substrate influences cellular immune function, the synthesis of inflammatory mediators, the generation of ROS, mitochondrial function, or combinations thereof. For example, in the case of arginine via excessive nitric oxide (NO) production and omega-3 fatty acids via eicosanoid synthesis, excessive amounts of these nutrients may have immunodepressant effects and may be associated with worse clinical outcomes. As another example, nutrients which further stimulate the systemic inflammatory response may be deleterious in critically ill patients. Without wishing to be bound by theory, critically ill patients appear to be characterized by hyperinflammation and cellular immune dysfunction coexisting in the same patient or patient population. Hence, for critically ill patients, nutrients that augment cellular defense (specific and non-specific immune function) and ameliorate reactive oxygen species without a collateral increase in the inflammatory response may be desired.

Certain examples of the present invention provide glutamine, antioxidants, or combinations thereof that may benefit critically ill patients by reducing or ameliorating, for example, hyperinflammation, cellular immune dysfunction, or oxidative stress. Furthermore, both critically ill patients as well as other patient groups, for example oncology patients or AIDS patients, may benefit from improvement of mitochondrial function.

In an aspect of the present invention there is provided a combination comprising glutamine from about 35 to about 380 grams or any range or amount therebetween per litre of solution provided as a short chain peptide and an antioxidant selected from the group consisting of selenium at a concentration from about 400 to about 10000 micrograms or any range or amount therebetween per litre, vitamin C at a concentration from about 1000 to about 20000 milligrams or any range or amount therebetween per litre, zinc at a concentration from about 20 to about 800 milligrams or any range or amount therebetween per litre, vitamin E at a concentration from about 500 to about 12000 milligrams or any range or amount therebetween per litre, and beta-carotene at a concentration from about 20 to about 4000 milligrams or any range or amount therebetween per litre. In certain examples, a combination may comprise glutamine and two or more antioxidants.

In certain examples, a combination comprising glutamine and an antioxidant may be delivered parenterally for treating critically ill patients, while in certain other examples the combination may be used for parenteral delivery to improve mitochondrial function.

Components of a combination do not have to be formulated within the same composition. In certain examples, a combination comprising glutamine and an antioxidant may be formulated in the same composition, while in other examples the glutamine and the antioxidant may be formulated in separate compositions. In still other examples, part of the glutamine and antioxidant dosage may be provided within the same composition, with the remaining dosage being provided in a separate composition.

Components of a combination do not have to be delivered using the same mode of administration. In certain examples, a combination comprising glutamine and an antioxidant may be delivered using the same mode of administration, while in other examples the glutamine and the antioxidant may be delivered using separate modes of administration. In one example, glutamine and an antioxidant are delivered parenterally. In another example, the glutamine is delivered parenterally, while the antioxidant is delivered enterally. In still other examples, part of the glutamine and antioxidant dosage may be provided with the same mode of administration (for example, parenteral), with the remaining dosage being provided in a separate mode of administration (for example, enteral).

In certain examples, a combination comprises glutamine and an antioxidant delivered simultaneously, while in other examples the glutamine and the antioxidant are delivered at separate times. In other examples, in treatment protocols that last several days, glutamine and an antioxidant may be delivered simultaneously during certain time periods, while being delivered at separate times during other time periods. Typically, glutamine and an antioxidant will be delivered on a daily basis (24 hour time period). However, for sake of convenience, efficacy, or practicality a person skilled in the art can easily deliver a combination of glutamine and an antioxidant on the basis of a different time period, for example, without limitation, a 72 hour, 48 hour, 36 hour, 24 hour, 12 hour, 6 hour, or 3 hour basis or any time period therebetween.

Among the many detrimental activities of ROS, or free oxygen radicals, is direct damage to mitochondrial DNA (mtDNA). Progressive accumulation of mtDNA damage can render cells unable to conduct oxidative phosphorylation reactions effectively, thereby leading to a bioenergetically deficient cell. Over time, mitochondrial DNA damage accumulates and leads to mitochondrial and cellular dysfunction with potential for subsequent organ failure, and ultimately death. Furthermore, in older patients a reduction in oxidant-protective enzymes superoxide dismutase and catalase are often observed. Accordingly, in many patients increases in the deleterious effects of ROS may be accompanied by a concommitant reduction in the enzymes and mitochondrial metabolites necessary for protection from ROS.

Mitochondria produce more than 90% of the energy needed to sustain mammalian life. Accordingly, mitochondrial dysfunction can impede the ability of cells to sustain and renew themselves, and may even lead to cell death.

Mitochondrial dysfunction is known to lead to a number of deleterious consequences including, without limitation, impaired calcium buffering, generation of free radicals, activation of the mitochondrial permeability transition and secondary excitotoxicity.

According to the United Mitochondrial Disease Foundation, Inc. (www.umdf.org) mitochondrial dysfunction appears to cause the most damage to cells of the brain, heart, liver, skeletal muscles, kidney and the endocrine and respiratory systems. Cells from long-lived tissue that have high energy demands such as neurons, pancreatic islet cells, cardiac and muscle cells may be particularly vulnerable to mitochondrial dysfunction. Depending on which cells are affected, symptoms may include loss of motor control, muscle weakness and pain, gastro-intestinal disorders and swallowing difficulties, poor growth, cardiac disease, liver disease, diabetes, respiratory complications, seizures, visual/hearing problems, lactic acidosis, developmental delays and susceptibility to infection.

Cellular degeneration associated with mitochondrial dysfunction is an important factor in various diseases. Mitochondrial dysfunction is known to be an important factor in several diseases including, without limitation, Progressive Infantile Poliodystrophy (Alpers Disease), NADH dehydrogenase (NADH-COQ reductase) deficiency (Complex I Deficiency), Ubiquinone-cytochrome c oxidoreductase deficiency (Complex III Deficiency), Cytochrome c oxidase deficiency caused by a defect in Complex IV of the respiratory chain (Complex IV Deficiency/COX Deficiency), Chronic Progressive External Opthalmoplegia Syndrome (CPEO), Kearns-Sayre Syndrome (KSS), Leber Hereditary Optic Neuropathy (LHON), Myoclonic Epilepsy and Ragged-Red Fiber Disease (MERRF), Neuropathy, Ataxia, and Retinitis Pigmentosa (NARP). Still other degenerative diseases are known to be associated with mitochondrial dysfunction, as indicated in US Patent Publication No. 20020173543 (filed Dec. 14, 2001) including, Alzheimer's Disease, diabetes mellitus, Parkinson's Disease, neuronal and cardiac ischemia, Huntington's disease and other related polyglutamine diseases, spinalbulbar muscular atrophy, Machado-Joseph disease (SCA-3), dentatorubro-pallidoluysian atrophy (DRPLA) and spinocerebellar ataxias, dystonia, Leber's hereditary optic neuropathy, schizophrenia, and myodegenerative disorders such as mitochondrial encephalopathy, lactic acidosis, and stroke (MELAS). Still further, mitochondrial dysfunction can be caused as a toxic side effect of therapeutic treatment, for example oncology treatments or antiretroviral treatments in AIDS/HIV patients. For example, most antibiotics (including, without limitation, tetracycline, erthyromycin, and chloramphenical) and anti-virals can cause mitochondrial dysfunction. Accordingly compositions, combinations, or unit dosage forms described herein may be useful in treating a variety of disorders of widely disparate genetic and acquired etiologies that have in common an association with mitochondrial dysfunction.

US Patent Publication No. 20020173543 describes various consequences of mitochondrial dysfunction including, without limitation, (i) decreases in ATP production, (ii) increases in the generation of highly reactive free radicals (e.g., superoxide, peroxynitrite and hydroxyl radicals, and hydrogen peroxide), (iii) disturbances in intracellular calcium homeostasis and (iv) the release of factors that initiate the apoptosis cascade. US Patent Publication No. 20020173543 describes several methods for assaying mitochondrial integrity including: (i) assay for Mitochondrial Permeability Transition (MPT) using 2-,4-Dimethylaminostyryl-N-Methylpyridinium (DASPMI); (ii) assay of apoptosis in cells treated with mitochondria protecting agents; and (iii) assay of Electron Transport Chain (ETC) activity in isolated mitochondria. As described herein, levels of mitochondrial and nuclear DNA may be compared to assay for mitochondrial function. Still other assays are known to the person skilled in the art.

The present inventor has found that glutamine, antioxidants, or combinations thereof may improve mitochondrial function and may benefit both critically ill patients as well as other patient groups, for example oncology patients or patients suffering from certain neurodegenerative diseases.

The amino acid glutamine plays a central role in nitrogen transport within the body, is a fuel for rapidly dividing cells particularly lymphocytes, is a precursor to glutathione, and has many other essential metabolic functions. Under normal physiological conditions glutamine is synthesized in large amounts by the human body and therefore it is considered non-essential.

Glutamine may become a conditionally essential amino acid in patients with catabolic disease. Several studies have shown that glutamine levels drop following extreme physical exercise, after major surgery (Blomqvist B I, Hammarqvist F, von der D, Wernerman J: Glutamine and alpha-ketoglutarate prevent the decrease in muscle free glutamine concentration and influence protein synthesis after total hip replacement. Metabolism 1995; 44:1215-1222), and during critical illness (Parry-Billings M, Evans J, Calder P C, Newsholme E A: Does glutamine contribute to immunosuppression after major burns? Lancet 1990; 336:523-525).

In animal studies, glutamine deprivation is associated with loss of intestinal epithelial integrity while glutamine supplementation decreases gut mucosal atrophy during total parenteral nutrition and preserves both intestinal and extra-intestinal IgA levels. However, with respect to bacterial translocation in animal models, studies of parenteral or enteral glutamine-supplemented formulas show mixed results. Some have shown decreased while others have demonstrated no such effect.

Glutamine supplementation has been suggested to benefit humans in maintaining gastrointestinal structure, decreasing intestinal permeability, preserving skeletal muscle, improving nitrogen balance, and enhancing immune cell function (Novak et al. Glutamine supplementation in serious illness: A systematic review of the evidence. Crit. Care Med 2002; 30; 2022-29). However, clinically significant doses and routes of administration have yet to be established for critically ill patients. Furthermore, the use of free L-glutamine in a clinical setting may be disadvantageous because of physical and chemical properties. First, glutamine is unstable during heat sterilization or prolonged storage due to cyclization and ammonia liberation. Second, free glutamine has a low solubility in water (approximately 36 g/L H₂O at 20 degrees Celsius), such that it is difficult to administer sufficient glutamine to critically ill patients, particularly patients that are volume restricted.

Certain examples of the present invention allow for greater concentrations of glutamine in the form of precursor glutamine molecules, than can be provided with the use of free glutamine alone. Accordingly the compositions of the present invention comprise a precursor glutamine molecule containing glutamine at a concentration from about 35 grams to about 380 grams or any range or amount therebetween per litre of solution. For example, glutamine concentration may be about 50 to about 150 grams per litre of solution. As another example, the concentration of glutamine may be about 35, 40, 45, 50, 55, 60, 65, 70, 75, 80, 85, 90, 95, 100, 110, 115, 120, 125, 130, 135, 140, 150, 160, 170, 180, 190, 200, 220, 240, 260, 280, 300, 320, 340, 360, or 380 grams per litre of solution or any concentration therebetween.

The upper limit of the glutamine concentration can be determined, among other factors, by the solubility properties of each specific precursor glutamine molecule. For example, an alanine-glutamine dipeptide has a solubility of 568 grams per litre at 20 degrees Celsius. A saturated solution of this dipeptide contains glutamine at a concentration of about 380 grams per litre. As another example, a saturated solution of a glycine-glutamine dipeptide (solubility of 154 grams per litre at 20 degrees Celsius) contains glutamine at a concentration of about 110 grams per litre.

The use of precursor glutamine molecules that have higher solubility than free glutamine allows for delivery of higher amounts of glutamine in smaller volumes than the volumes of 1.5 to 2 litres that are associated with glutamine supplemented total parenteral nutrition. A unit dosage form of the invention will typically have a total volume from about 50 to about 1000 millilitres or any range or amount therebetween. For example, the total volume may be from about 200 to about 500 millilitres. As another example, the total volume of the unit dosage form may be about 500, 450, 400, 350, 300, 250, 200, 150, 100, or 50 millilitres or any volume therebetween.

Thus, the present invention benefits critically ill patients by achieving effective doses of glutamine in smaller volumes. An example of an effective dose is a daily dose of about 0.3 g glutamine/kg body weight to about 0.9 g glutamine/kg body weight or any range or amount therebetween. Further examples include a daily dose of about 0.4 g glutamine/kg body weight to about 0.8 g glutamine/kg body weight, or about 0.5 g glutamine/kg body weight to about 0.7 g glutamine/kg body weight, or a range beginning with any dose therebetween, including without limitation 0.35, 0.4, 0.45, 0.5, or 0.55 g glutamine/kg body weight.

Glutamine can be liberated from precursor glutamine molecules within a patient's body. Examples of precursor glutamine molecules include, without limitation, glutamine derivatized with alkyl, carboxy, acetyl, ester, or amide groups. A preferred form of a glutamine precursor molecule is a short chain peptide containing glutamine. The length of the short chain peptide is preferably from two residues to about 10 residues, including for example dipeptides or tripeptides. Mixtures of short chain peptides comprised of varying residues and of varying residue length are contemplated. For example, a composition of the invention may comprise a short chain peptide selected from alanine-glutamine-glutamine, glycine-glutamine-glycine, glycine-glutamine-glutamine, glycine-glutamine, alanine-glutamine, arginine-glutamine, proline-glutamine, serine-glutamine, valine-glutamine, and any combination thereof. Accordingly, a desired high glutamine concentration is achieved, without requiring a similarly high concentration of another amino acid.

In addition, to various types of short chain peptides containing glutamine being used together, a single type of glutamine precursor or any combination of different glutamine precursors may be used, provided that the total glutamine concentration is sufficiently high for treatment of critically ill patients. For example, alpha-ketoglutarate could be combined with a short-chain peptide containing glutamine. As another example, a carboxy derivatized glutamine precursor molecule, could be combined with free glutamine and a mixture of short chain peptides containing glutamine ranging from two to five residues in length.

Persons skilled in the art will recognize various sources for obtaining short chain peptides containing glutamine. For example, an enzyme-catalyzed dipeptide synthesis may be accomplished using an N-protected amino acid ester as an electrophile, a free glutamine as a nucleophile, and a plant ficin protease (Furst P: New developments in glutamine delivery, Amer Soc Nutritional Sci 2001:2562s-2568s). Furthermore, short chain peptides containing glutamine may be isolated from hydrolysates of proteins that are naturally rich in glutamine, for example a carob protein (U.S. Pat. No. 5,849,335 issued Dec. 15, 1998). Even further, short chain peptides containing glutamine can be obtained from transgenic cells or organisms engineered to produce a protein that has repeating units of glutamine-containing sequences separated by recognized protease cleavage sites (U.S. Pat. No. 6,649,746 issued Nov. 18, 2003)). Furtherstill, dipeptides containing glutamine are commercially available, for example Dipeptiven®, Glamin®, and Intestamin® (Fresenius Kabi, Uppsala, Sweden).

Dipeptiven® is a 20%-solution of the glutamine-containing dipeptide, N(2)-L-alanyl-L-glutamine. 100 mls of Dipeptiven® contains 20 g N(2)-L-alanyl-L-glutamine (=8.20 g L-alanine, 13.46 g L-glutamine). The dipeptide is highly soluble in water (568 g/L H₂0 at 20° C.) and remains stable during heat sterilization and storage. In contrast, the physical and chemical properties of free glutamine (low solubility, 36 g/L H₂0 at 20° C., and poor storage characteristics) hamper its use in aqueous solutions. Therefore the ala-gln dipeptide, which does not have the disadvantages of free glutamine, is a non-limiting example of a short chain peptide that may serve as a precursor for free glutamine in clinical settings.

Dipeptiven® may be administered parenterally, with intravenous infusion preferred. After intravenous infusion, the dipeptide ala-gln is rapidly hydrolysed into the amino acids L-glutamine and L-alanine. This is ensured due to high peptidase activity, existing nearly in all body compartments. Several studies have demonstrated the hydrolysis of the ala-gln dipeptide in humans following intravenous administration by measuring the quantities of free alanine and glutamine. After bolus injections of different amounts of ala-gln in healthy volunteers, a half-life between 2.4 and 3.8 minutes and a plasma clearance rate of 1.92 l/minute have been reported. The calculated distribution volume was equivalent to that of the extracellular compartment. Following a continuous 4-hour infusion of 24 mg ala-gln/kg body weight/hour in healthy volunteers, a rapid equimolar rise of glutamine and alanine plasma concentrations was observed. Over the infusion period only trace quantities of ala-gln were found in plasma. Fifteen minutes after the end of infusion, the dipeptide was no longer detectable in plasma. Additionally, no ala-gln was detected in urine. Concentrations of other amino acids were not affected. The ala-gln solutions were well tolerated and no subjective discomfort was reported (Albers S, Wernermann J, Stehle P, Vinnars E, Fürst P. Availability of amino acids supplied by constant intravenous infusion of synthetic dipeptides n healthy volunteers. Clinical Science 1989; 76:643-648).

Glamin® is an amino acid solution containing 30.27 g/L of glycyl-L-glutamine (gly-gln). Like the ala-gln dipeptide, gly-gln remains stable during heat sterilization and storage. Solubility of gly-gln (154 g/L H₂0 at 20 degrees Celsius) is less than that of ala-gln (568 g/L), but approximately 5-fold higher than that of free glutamine (36 g/L). Glamin® may be administered parenterally, with an intravenous route being preferred.

In certain examples, the compositions of the present invention may be used in combination with parenteral or enteral supplements. For example, Intestamin® is an enteral supplement containing glutamine as dipeptides, and antioxidants (per 100 ml, Intestamin contains 60 ug of selenium, 4 mg of Zinc, 2 mg of B-carotene, 100 mg of Vitamin E, and 300 mg of Vitamin C). The recommended daily dosage (RDD) delivers nutrients within a volume of 500 ml. Consequently the product has been designed for the dietary management of critically ill patients with limited enteral tolerance and in need of glutamine and antioxidant supply. The RDD of 500 ml delivers 30 g glutamine provided as dipeptides. Intestamin® is used as an enteral supplement to parenteral or enteral nutrition.

Results from a randomised clinical trial show that the application of Intestamin® is safe and well tolerated in patients with severe pancreatitis. Moreover, results from several observational studies show that the enteral glutamine containing supplement was well tolerated in the early postoperative setting and that a large dose of micronutrient supply with Intestamin® was associated with the correction of the low postoperative plasma values within 5 days (Berger M M, Goette J, Stehle P, Cayeux M, Chioléro R, Schroeder J. Enteral absorption of a solution with high dose antioxidants and glutamine early after upper gastrointestinal surgery. Clin Nutr 2002, Vol. 21, Supplement 1, p 17).

An aspect of the present invention relates to administration of antioxidants, for example, without limitation, selenium, vitamin C, zinc, vitamin E, beta-carotene, or combinations thereof.

Selenium is an essential co-factor in glutathione enzymatic function and has favorable effects on cellular immune function. Selenium may have additional impact through other selenoproteins containing seleno-cysteine. There are about 20 known selenium-containing proteins in mammals. Selenium is inserted into protein as the amino acid seleno-cysteine. These proteins have a series of newly discovered antioxidant activities, i.e. redox stabilizing properties, including regulation of gene expression.

The recommended daily allowances for elemental selenium as reported in the Pharmacological Basis of Therapeutics, Ninth Edition, page 1540, The McGraw-Hill Companies, 1996 ranges from 10 to 75 micrograms per day. Higher selenium doses may have a significant effect in treatment of critically ill patients or for improving mitochondrial function. The present invention comprises administering a daily dose from about 400 to about 2000 micrograms per day to critically ill patients or patients suffering from mitochondrial dysfunction. Doses of about 400, 500, 600, 700, 800, 900, 1000, 1100, 1200, 1300, 1400, 1500, 1600, 1700, 1800, 1900, or 2000 micrograms per day or any dose therebetween may be useful.

Selenium may be incorporated into the compositions, combinations or unit dosage forms of the invention as elemental selenium or a non-toxic organic or inorganic salt, chelate or other selenium compound as a precursor of elemental selenium. In the compositions, combinations or unit dosage forms of this invention, selenium may be employed as one of several non-toxic, organic or inorganic selenium compounds capable of being absorbed by the body.

Examples of inorganic selenium compounds are aliphatic metal salts containing selenium in the form of selenite or selenate anions. Organic selenium compounds are typically less toxic than inorganic compounds. Non-limiting examples of organic selenium compounds include selenium cystine, selenium methionine mono- and di-seleno carboxylic acids with about seven to eleven carbon atoms in the chain. Seleno amino acid chelates may also be used. Further, seleno compounds that are commercially available may be used.

The compositions, combinations or unit dosage forms of the present invention comprise selenium in high concentrations in order to provide effective doses to patients with restricted volume requirements. For example, selenium may be at a concentration from about 400 microgram to about 10000 microgram or any range or amount therebetween per litre of solution. For example, selenium concentration may be from about 1000 to about 4000 micrograms per litre. Further examples of selenium concentrations include, without limitation, concentrations of about 400, 600, 800, 1000, 1200, 1400, 1600, 1800, 2000, 2200, 2400, 2600, 2800, 3000, 3200, 3400, 3600, 3800, 4000, 4500, 5000, 5500, 6000, 6500, 7000, 8000, 9000, or 10000 micrograms per litre of solution or any concentration therebetween.

Selenium is a non-limiting example of an antioxidant that may be used in combination with a precursor glutamine molecule for treating critically ill patients or for improving mitochondrial function. The compositions, combinations or unit dosage forms of the present invention may comprise any type of antioxidant without limitation. For example, an antioxidant may be selected from the group consisting of selenium, beta-carotene, vitamin E, vitamin C, zinc, and any combination thereof. As with the exemplification of selenium, the present invention contemplates other antioxidants at doses greater than recommended dietary allowances (RDA) or tolerable upper intake level (UL) for healthy individuals, in order to achieve a significant clinical effect in critically ill patients or patients suffering from mitochondrial dysfunction. RDA and UL values are determined, for example, in Dietary Reference Intake reports that are published by The National Academies (reports may be accessed via www.nap.edu). The present invention comprises antioxidants at concentrations that are at least greater than RDA levels. In certain non-limiting examples antioxidant concentration is greater than the UL.

Beta-carotene is naturally occurring provitamin A with lipid antioxidant properties. In addition, beta-carotene is lipid soluble and is concentrated in circulating lipids. In vitro, beta-carotene is an unusual type of chain breaking lipid antioxidant. Because of its many conjugated double bonds, beta-carotene exhibits good radical trapping antioxidant behavior. In the context of the present invention beta-carotene may be provided in a dose ranging from 10 mg to 1000 mg per day, or any range or dose therebetween.

Vitamin E (alpha-tocopherol) is a fat soluble vitamin. Its primary function is as a lipid antioxidant protecting lipids from oxidative modification. Water-soluble derivatives of vitamin E (for example, as disclosed in U.S. Pat. Nos. 6,022,867 and 6,645,514) are known and may be used in a water based composition. Furthermore, stable water miscible emulsions may also be used to increase the solubility of vitamin E. In the context of the present invention Vitamin E may be provided in a dose ranging from 500 mg to 3000 mg per day, or any range or dose therebetween.

Vitamin C is a water-soluble antioxidant that is critical for the production of collagen, and therefore needed in wound healing. Further, Vitamin C helps protect the fat-soluble vitamins A and E as well as fatty acids from oxidation. Vitamin C is also involved in iron absorbtion. In the context of the present invention Vitamin C may be provided in a dose ranging from 1000 mg to 5000 mg per day, or any range or dose therebetween.

Zinc is an essential trace mineral that has antioxidant properties. Zinc plays a critical role in cellular biology, and is involved in virtually every important cellular process such as transcription, translation, ion transport, and others. In the context of the present invention zinc may be provided in a dose ranging from 20 mg to 200 mg per day, or any range or dose therebetween.

In humans, there is a complex endogenous defence system designed to protect tissues from reactive oxygen species (ROS) or reactive nitrogen-oxygen species (RNOS) induced cell injury. Special enzymes such as superoxide dismutase, catalase, and glutathione peroxidase (including their co-factors selenium, zinc, manganese, and iron), sulfhydryl group donors (i.e. glutathione), and vitamins (including, but not limited to vitamin E, C, and β-carotene) form a network of functionally overlapping defence mechanisms. In critically ill patients, there is increasing evidence that these defence mechanisms can be overwhelmed due to the local or systemic imbalance between increased ROS/RNOS production and a reduced capacity for elimination. Further, numerous studies provide evidence that low endogenous “stores” of antioxidants are associated with an increase in free radical generation, an augmentation of the systemic inflammatory response, subsequent cell injury, increased morbidity and even higher mortality in the critically ill (Alonso de Vega J M, Diaz J, Serrano E, et al. Oxidative stress in critically ill patients with systemic inflammatory response syndrome. Crit Care Med 2002; 30:1782-1786). The present invention provides an effective dose of exogenous antioxidant micronutrients to counteract the depletion of the circulating antioxidants and thereby counteract the overzealous production of toxic oxygen free radicals that can cause mitochondrial dysfunction. Furthermore, the exogenous antioxidants may optionally be provided outside the context of enteral or parenteral nutrition, for example, in the absence of carbohydrates or lipids.

Therefore, certain examples of the present invention provide a composition, combination or unit dosage form comprising glutamine from about 35 to about 380 grams or any range or amount therebetween per litre of solution provided as a short chain peptide, and an antioxidant selected from the group consisting of selenium at a concentration from about 400 to about 10000 micrograms or any range or amount therebetween per litre, vitamin C at a concentration from about 1000 to about 20000 milligrams or any range or amount therebetween per litre, zinc at a concentration from about 20 to about 800 milligrams or any range or amount therebetween per litre, vitamin E at a concentration from about 500 to about 12000 milligrams or any range or amount therebetween per litre, and beta-carotene at a concentration from about 20 to about 4000 milligrams or any range or amount therebetween per litre. In some examples, the antioxidant is selenium at a concentration, of about 1000 to about 4000 micrograms or any range or amount therebetween per litre, and the concentration of glutamine is from about 50 to about 150 grams or any range or amount therebetween per litre of solution. In some examples, the composition, combination or unit dosage form may be delivered parenterally to a critically ill patient or a patient suffering from mitochondrial dysfunction. Furthermore, lipids, carbohydrates or both lipids and carbohydrates are optionally absent. This composition, combination or unit dosage form may be formulated in a much lower volume than currently available parenteral nutrient compositions, and may be used for administration to volume-restricted patients.

Certain examples of the present invention pertain to use of a composition, combination or unit dosage form comprising high concentrations of glutamine, antioxidants, or combinations thereof for treating critically ill patients or patients suffering from mitochondrial dysfunction. Accordingly, treatment comprises administering a composition, combination or unit dosage form comprising glutamine from about 35 to about 380 grams or any range or amount therebetween per litre of solution, for example greater than 35 grams per litre of solution, provided as a short chain peptide, and an antioxidant selected from the group consisting of selenium, vitamin C, zinc, vitamin E, beta-carotene, and combinations thereof, to a critically ill patient in need of such treatment or to improve mitochondrial function in a patient suffering from mitochondrial dysfunction.

In some examples, the compositions or combinations of the invention may be prepared in unit dosage forms for ease of administration. A unit dosage form is a convenient amount of glutamine and antioxidants for treatment of critically ill patients or patients suffering from mitochondrial dysfunction, that can be administered to a patient as part of a regular regime. The unit dosage form can be in any convenient form including, without limitation, dry solid, lyophilized powder, freeze-dried, or liquid. For example, a composition comprising a glutamine and an antioxidant could be stored as an individual measured solid that could then easily be dissolved in an appropriate volume of saline solution prior to administration. As another example, a combination comprising glutamine and an antioxidant could be packaged and stored in two separate premeasured volumes that could then be directly administered to patients.

The compositions, combinations, or unit dosage forms of the present invention are prepared according to conventional techniques adopted in the preparation of pharmaceutical forms for parenteral use. While the compositions and unit dosage forms of the present invention are typically formulated for parenteral delivery, other modes of administration may be used to achieve increased delivery of glutamine, antioxidants, or combinations thereof. The compositions of the present invention are preferably administered in liquid form with a unit dosage form having less than 1000 millilitres of volume. For example, volume is about 1000, 900, 800, 700, 600, 500, 450, 400, 350, 300, 250, 200, 150, 100 or 50 millilitres or any volume therebetween. As another example, volume is from about 50 millilitres to about 500 millilitres, or any range or volume therebetween.

As described in the Examples, compositions, combinations, or unit dosage forms of the present invention have been administered to patients and several benefits have been observed. For example, a combination comprising glutamine and selenium was shown to improve mitochondrial function, using an assay that monitors levels of mitochondrial DNA (mtDNA) relative to nuclear DNA (nDNA), in patients. In other examples, patients appear to demonstrate improved resolution of oxidative or free radical damage as indicated, for example, by a reduction in markers of oxidative stress, and preservation of glutathione levels. In still other examples, a combination comprising glutamine and an antioxidant may be administered to patients without any apparent adverse effect on organ function or levels of inflammatory cytokines.

The present invention will be further illustrated in the following examples.

### EXAMPLES

### Example 1

### Compatibility of Selenious Acid in Dipeptiven®/Normal Saline Admixtures

Delivering nutrients in large-volume solutions, like total parenteral nutrition, limits the utility in volume restricted patients. Therefore, to enhance the clinical application of a Glutamine and Selenium combination, there was a need to determine whether this combination could be provided in small volumes. There was a concern that selenious acid may be reduced to elemental selenium, which is insoluble and may form particulate matter. Hence, the compatibility of providing glutamine dipeptides with selenium by continuous intravenous infusions was evaluated.

A. Test Design

1. Test Preparations

The following test preparation were used
a) MicroSe 40 μg/ml, Baxter 10 ml, Lot 120669 (Selenium acid USP)
b) Dipeptiven Fresenius Kabi 100 ml, Lot SD 1667 (20% L-Alanyl-L-Glutamine (Ala-Gln) in water for injection, pH 5.4-6.0)

Dipeptiven® is a 20%-solution of the glutamine-containing dipeptide, N(2)-L-alanyl-L-glutamine (AlaGln). One hundred mls of Dipeptiven® contains 20 g N(2)-L-alanyl-L-glutamine (=8.20 g L-alanine, 13.46 g L-glutamine). The dipeptide is highly soluble in water (568.0 g/l H20 at 20° C.) and remains stable during heat sterilization and storage. In contrast, the physical and chemical properties of free glutamine (which has limited solubility and poor storage characteristics) hamper its use in aqueous solutions. Therefore the AlaGln dipeptide, which does not have the disadvantages of free glutamine, serves as a precursor for free glutamine in clinical settings. There are 0.7 grams of free glutamine per gram of Dipeptiven® yielding a total dose of glutamine of 0.35 grams/kg/day.

The selenium used in this study was a Selenious acid injection (MICRO Se(g, Sabex Inc Quebec, Canada). It is indicated as a supplement to intravenous solution given for total parenteral nutrition. Each ml of MICRO Se contains 65.36 μg of Selenious acid (equivalent to 40 μg Selenium/ml).

2.Container and Carrier Solutions

The following carrier solutions and containers were used
a) 250 ml 0.9% NaCl USP, Lot W4H16C0, Baxter (PVC-bags)
b) 250 ml 0.9% NaCl USP, Lot J4H721, B. Braun (non-PVC-bags)
c) 500 ml 0.9% NaCl USP, Lot W4H09B1, Baxter (PVC-bags),
d) 500 ml 0.9% NaCl USP, Lot J4H636, B. Braun (non-PVC-bags)

3. Test Admixtures, Their Preparation, Storage Conditions and Sampling

Test admixtures were prepared under Laminar flow conditions by extracting from the 0.9% NaCl USP solutions in bags with sterile syringes via the injection port the respective volumes to be added later on of Dipeptiven. These quantities of the extracted normal saline solutions were discarded and the same volumes of Dipeptiven were added to the remaining part of 0.9% NaCl USP in the bags by sterile syringes via the injection port.

After these steps, 12.5 ml (approx. 500 μg Selenium) MicroSe (40 μg/ml) were added by a sterile syringe.

Two different bag qualities in two different sizes of 250 ml and 500 ml were used, non-PVC-Polyolefin-bags and PVC-bags.

The composition of the resulting test samples is given below in Table 1.

TABLE 1
Composition of test samples

| | Volume | | Volume | Theoretical |
|---|---|---|---|---|
| | 0.9% NaCl | Volume | MiroSe | osmolarity* |
| Bag size/material | USP | Dipeptiven | 40 μg/ml | of mixture |
| 250 ml/PVC | 125 ml | 125 ml | 12.5 ml | 585 |
| 250 ml/non-PVC | 50 ml | 200 ml | 12.5 ml | 760 |
| 500 ml/PVC | 375 ml | 125 ml | 12.5 ml | 450 |
| 500 ml/non-PVC | 250 ml | 250 ml | 12.5 ml | 600 |

*Values are calculated based on the theoretical osmolarity for 0.9% NaCl USP of 308 mOsm/l and for Dipeptiven of 921 mOsm/l under the assumption of additivity of volumes.

Sampling times were 0, 24, 48, 72 and 96 hours after storage at room temperature.

Samples for Selenium assay were filtrated by 0.22 μm filters to eliminate possible Selenium precipitates.

Additional stress admixtures with excessive quantity of Selenium were prepared with the following compositions as indicated in Table 2.

TABLE 2
Composition of stress admixtures

| | Volume | Volume | Volume |
|---|---|---|---|
| Bag size/material | 0.9% NaCl USP | Dipeptiven | MiroSe 40 μg/ml |
| 500 ml/PVC | 50 ml | 200 ml | 125 ml |
| 500 ml/non-PVC | 50 ml | 200 ml | 125 ml |

Test period: after 0, 96 hours

Storage conditions: room temperature

For every composition 2 bag samples were analysed.

4. Test Parameters and Methods

The following test parameters and methods were used
Appearance according Ph. Eur.
Discoloration according Ph. Eur.
UV-Absorption E 4/400 (extinction in a 4 cm cell at 400 nm)
Subvisible particulate matter according Ph. Eur.
pH according Ph. Eur.
L-Alanyl-L-Glutamine assay according method AP-S542 (according Fresenius Kabi AP-542)
Selenium assay (atomic absorption method according USP)

4.1 Appearance According Ph. Eur.

The tests were executed with regard to visible particulate matter, opalescence/opacity (acc. Ph. Eur.; European Pharmacopeia, 8th Edition. Strasbourg: Council of Europe; 2005), precipitation and gas bubble generation.

4.2 Discoloration According Ph. Eur.

Samples from the test solutions were compared to standard colour solutions according Ph. Eur.

4.3 UV-Absorption E 4/400 (Extinction in a 4 cm Cell at 400 nm)

The determination was performed by an UV/VIS-double-beam spectrophotometer (Hitachi U-2000), corresponding to the Ph. Eur. A 4 cm quartz glass cuvette was used to measure the absorbance at 400 nm. The measurement was carried out against water as reference solution.

4.4 Subvisible Particulate Matter According Ph. Eur.

The examination corresponds to Ph. Eur. light blockage methode and was executed with the particle-counter model 9064 (HIAC-ROYCO). The quantity of particles ≧10 microm/ml and ≧25 microm/ml was determined. The solution meets the requirements of the test if the average numbers of particles present in the units tested do not exceed 25 counts/ml ≧10 microm and 3 counts/ml ≧25 microm.

4.5 pH According Ph. Eur.

The determination of pH was carried out using a pH-meter (pH-Meter 761 Calimatic, Knick) corresponding to the Ph. Eur.

4.6 L-Alanyl-L-Glutamine (AlaGln) Assay

L-Alanyl-L-Glutamine (AlaGln) levels were determined by HPLC.

4.7 Selenium Assay (Atomic Absorption Method According USP)

Selenium was determined according USP with an atomic absorption method.

To exclude interference with the organic matrix of the samples the Hydride Method variant was selected.

Description of Method

Selenium is reduced to the Hydride under Nitrogen with alkaline Sodium Boron Hydride and transferred to the sample cell of the atomic absorption spectrometer. Measurement is executed at 196.0 nm and a split of 2.0 nm.

Atomic absorption spectrometer 272, Hydride-system MHS-1 and lamp EDL all from Perkin Elmer were used.

Reagents
Sodium Borohydride p.A. Merck (product-no. 106371)
Sodium Hydroxide p.A. Merck (product-no. 106495)
Hydrochloric acid 37% p.A. Merck (product-no. 100317)

1.5% Sodium Borohydride in 3% Sodium Hydroxide and 3% Hydrochloric acid were prepared from the reagents with distilled water.

Sample Preparation

30 μl of the admixtures in the 250 ml bags and 60 μl of the admixtures in the 500 ml bags were added to 5 ml 1.5% HCl. The stress samples were diluted before with water 1:10. To this sample solution an excess of the alkaline Boron Hydride solution was added according the recommended procedure of Perkin Elmer.

Standard Preparation

MicroSe 40 μg Baxter Lot 120669, which was the lot to be tested for stability was also used as a standard. Content of Selenium of this product was tested before running the suitability tests using a second independent working standard. Compliance with the declaration according USP (95-105% of declaration) was confirmed with 105% of Selenium declaration.

Suitability of Method Under Actual Condition of Use

To the admixtures of Dipeptiven and 0.9% NaCl USP according point 3. quantities of 450, 500 and 550 μg Selenium (in form of 11.25, 12.50 and 13.75 ml of Microselen 40 μg/ml) were added to check the recovery of the method at 90%, 100% and 110% of the Selenium addition quantities according to point 3.

The results of the measurements (mean of triplicates and standard deviations) are given in Table 3 below.

Results

The results from Table 3 show recovery between 102-111% in the admixtures and standard deviation between 3.3-6.3%. This is satisfactory for an analytical method for a trace element in a diluted organic matrix.

TABLE 3
Recovery from admixtures.

| | | added Selenium | | |
|---|---|---|---|---|
| Admixture | | 450 μg | 500 μg | 550 μg |
| 125 ml NaCl + 125 ml | x* | 491.7 | 537.5 | 607.5 |
| Dipeptiven (1:1) | sᵣₑₗ | 3.3 | 4.5 | 4.3 |
| | recovery | 109.2% | 107.5% | 110.5% |
| 50 ml NaCl + 200 ml | x* | 487.1 | 535.8 | 601.7 |
| Dipeptiven (1:4) | sᵣₑₗ | 3.4 | 3.3 | 3.6 |
| | recovery | 108.2% | 107.1% | 109.3% |
| 375 ml NaCl + 125 ml | x* | 496.7 | 523.3 | 559.2 |
| Dipeptiven (3:1) | sᵣₑₗ | 3.2 | 5.4 | 4.8 |
| | recovery | 110.4% | 104.0% | 101.6% |
| 250 ml NaCl + 250 ml | x* | 500.4 | 552.1 | 566.3 |
| Dipeptiven (1:1) | sᵣₑₗ | 6.3 | 4.1 | 3.8 |
| | recovery | 111.2% | 104.4% | 102.0% |

*x = average

4.8 Time Course Testing

Preparation of the Study Solutions, Storage Condition and Sampling

The study protocol uses different ranges of high and low concentrations of AlaGln and selenium to cover examples of concentrations that are thought to be clinically relevant. Two different intravenous bags sizes (250 ml and 500 ml) of normal saline (0.9% NaCl) were used. Both Polyvinyl Chloride (PVC) and non-PVC bags were used for all scenarios. The volume of Dipeptiven® to be added to the bags was first extracted and then replaced 125 ml and 200 ml of Dipeptiven® for the 250 ml bags and 125 and 250 ml of Dipeptiven® for the 500 ml bags. Following these steps, 500 μg Selenium (12.5 ml Micro Se®) were added to every bag. In addition to these standard admixtures, we added excessive quantities of selenium (125 ml of Micro Se®, 200 ml of Dipeptiven®, and 50 ml of saline) to further assess the stability of high dose selenium and one solution with no normal saline (250 ml of Dipeptiven® and 12.5 ml Micro Se® only). All test solutions were prepared under Laminar flow conditions using sterile conditions.

Samples were then stored at room temperature for a period of 0, 24, 48, 72 and 96 hours of time at which time the following observations and tests were conducted.

Test Parameter and Methods

All testing was conducted according to the European Pharmacopoeia (Ph. Eur.; European Pharmacopeia, 8th Edition. Strasbourg: Council of Europe; 2005) which sets out the common standards for the composition of substances used in the manufacture of medicines. The study mixtures were carefully examined for visible particulate matter, opalescence/opacity, precipitation, discoloration, and gas bubble generation. Mixtures were also examined for subvisible particulate matter using the light blockage method and with a particle-counter model 9064 (HIAC-ROYCO). The quantity of particles ≧10 microm/ml and ≧25 microm/ml was determined. As specified by Ph. Eur., the solution meets the requirements of the test if the average numbers of particles present in the units tested do not exceed 25 counts/ml ≧10 microm and 3 counts/ml ≧25 microm. To quantify discoloration, samples were subjected to UV-Absorption E 4/400 (extinction in a 4 cm cell at 400 nm) using a UV/VIS-double-beam spectrophotometer (Hitachi U-2000). Therefore, a 4 cm quartz glass cuvette was used to measure the absorbance at 400 nm using water as reference solution.

pH of all solutions was determined using a pH-meter (pH-Meter 761 Calimatic, Knick) corresponding to the Ph. Eur. Finally, AlaGln concentration was determined using the High Performance Liquid Chromatography (HPLC) method corresponding to the laboratory's standard procedure. The glutamine dipeptides were assayed by an isocratic reversed Phase C18-HPLC method with UV detection at 214 nm with a Potassium dihydrogen Phosphate-buffer (0.05 molar) as mobile phase. The samples for selenium level determination were first filtrated by 0.22 μm filters to eliminate possible selenium precipitates and then assayed using the atomic absorption method. To exclude interference with the organic matrix of the samples the Hydride Method variant was selected according to Ph. Eur. The content of AlaGln and selenium were determined twice at each time point and the results are presented as average values. The initial or baseline value (Time=0) was set equal to 100%.

Results

There was no evidence of turbidity, discoloration, or subvisible particulate matter for any admixture at any time point. The pH was stable across time for all solutions. The concentration of AlaGln and selenium did not change significantly over time with any of the admixtures (see Table 4).

TABLE 4
Results of Assays for Dipeptiven ® (AlaGln) and Selenium (%)

| | | Time (hours) | | | | |
|---|---|---|---|---|---|---|
| Admixture | Nutrient | 0 | 24 | 48 | 72 | 96 |
| 125 ml of 0.9% NaCl + | AlaGln | 100 | 101 | 101 | 98 | 101 |
| 125 ml of Dipeptiven ® + | Se | 100 | 100 | 98 | 103 | 100 |
| 12.5 ml of Se | | | | | | |
| 50 ml of 0.9% NaCl + | AlaGln | 100 | 104 | 102 | 97 | 103 |
| 200 ml of Dipeptiven ® + | Se | 100 | 104 | 105 | 106 | 100 |
| 12.5 ml of Se | | | | | | |
| 375 ml of 0.9% NaCl + | AlaGln | 100 | 101 | 101 | 98 | 101 |
| 125 ml of Dipeptiven ® + | Se | 100 | 104 | 103 | 107 | 100 |
| 12.5 ml of Se | | | | | | |
| 250 ml of 0.9% NaCl + | AlaGln | 100 | 100 | 101 | 99 | 101 |
| 250 ml of Dipeptiven ® + | Se | 100 | 102 | 98 | 103 | 101 |
| 12.5 ml of Se | | | | | | |
| 50 ml of 0.9% NaCl + | AlaGln | 100 | — | — | — | 95 |
| 200 ml of Dipeptiven ® + | Se | 100 | — | — | — | 100 |
| 125 ml of Se | | | | | | |
| 0 ml of 0.9% NaCl + | AlaGln | 100 | — | — | — | 97 |
| 250 ml of Dipeptiven ® + | Se | 100 | — | — | — | 101 |
| 12.5 ml of Se | | | | | | |

Legend:
NaCl—Sodium Chloride;
AlaGln—N(2)-L-alanyl-L-glutamine;
Se—Selenium
All results are reported in non polyvinyl chloride (PVC) bags. Results are similar in PVC bags.
— not done.

The stability study protocol covers various examples admixtures of various concentrations of both active ingredients Selenium and L-Alanyl-L-Glutamine and also takes into consideration the size/volume as well as different materials of the primary packaging material.

This compatibility study shows that in the intended admixture/dosage range of the clinical study the active ingredients L-Alanyl-L-Glutamine and Selenium are stable and the general specifications for large volume parenterals are met of all samples over test period and do not change significantly over 96 hours at room temperature storage.

No differences are seen between the two primary bag systems PVC-bag and non PVC-bat nor any dependence on the volume/size of the bags 250 ml and 500 ml were recorded.

The results indicate that the active ingredients AlaGln and Selenium when combined do not result in any changes to the physical or chemical nature of the nutrients over 96 hours at room temperature storage. No differences are seen between the two primary bag systems, PVC-bag and non PVC-bag, nor were there any differences between the size of the bag or volume of normal saline. Glutamine dipeptides (AlaGln) and selenium appear compatible in solution for up to 96 hours when stored at room temperature. By providing the study nutrients as a single intravenous administration, there is reduced the risk of error related to the administration of these nutrients to patients.

### Example 2

### Administration of Glutamine Dipeptides and Antioxidants in Critically Ill Patients

Study Design: A single center, open-label, phase I dose ranging clinical trial with prospective controls

Setting: Kingston General Hospital (KGH), a tertiary care ICU in Ontario, Canada.

Study population: Mechanically ventilated adult patients (≧8 years old) admitted to ICU with clinical evidence of hypoperfusion. Underweight (<50 kgs) patients and those with severe head trauma (GCS <8 or need for ventriculostomy) are excluded due to safety reasons.

Clinical evidence of hypoperfusion is defined as:
need for vasopressor agents (norepinephrine, epinephrine, neosynephrine, ≧5 mg/kg/min of dopamine or vasopressin) for more than one hour; or
a systolic blood pressure ≦90 mmHg or the mean arterial pressure <70 mmHg for more than one hour despite adequate fluid challenge; or
unexplained metabolic acidosis with a pH ≦7.30 or a base excess ≧5.0 in association with an elevated a blood lactate concentration (≧4 mmol/l).

Study Intervention: As summarized in Table 5, patients were sequentially enrolled into one of 5 groups:
Group 1: 30 patients who meet study eligibility criteria to determine the baseline rate of study measurements including adverse events, organ function, and need for dialysis. This group received no glutamine/selenium but the same routine clinical and biochemical measurements were taken in this group as with subsequent groups with the exception of serum ammonia, amino acid levels, glutathione peroxidase and other mechanistic markers (IL-18, TBARS, etc.). These measurements were be used to determine the baseline rate of study measurements including adverse events, organ function, and need for dialysis.
Group 2: The next 7 patients received a standard dose of Dipeptiven®, 0.5 gms/kg/day of glutamine dipeptides (0.35 grams/kg/day of glutamine) intravenously and nothing enterally
Group 3: The next 7 patients received Dipeptiven®, 0.5 gms/kg/day of glutamine dipeptides (0.35 grams/kg/day of glutamine) intravenously and 21.25 grams/day of glutamine dipeptides (15 grams/day of glutamine) and 150 microg of selenium enterally provided as 250 ml of Intestamin® (indicated as “½ can” in Table 5) per day via nasogastric tube infusion;
Group 4: The next 7 patients received Dipeptiven®, 0.5 gms/kg/day of glutamine dipeptides (0.35 grams/kg/day of glutamine) intravenously and 42.5 grams/day of glutamine dipeptides (30 grams/day of glutamine) and 300 microg of selenium enterally provided as 500 ml of Intestamin® (indicated as “full can” in Table 5) per day via nasogastric tube infusion;
Group 5: The next 7 patients receive the same doses of Dipeptiven® parenterally and Intestamin® enterally (indicated as “full can” in Table 5) as group 4 but receive an additional 500 microg of selenium parenterally (800 microg in total).

TABLE 5
Summary of Study Intervention.

| | | Dose of Dipeptides (gm/kg/day) | | |
|---|---|---|---|---|
| Group | N | Parenterally* | Enterally\hat{ } | AOX |
| 1 | 30 | 0 | 0 | 0 |
| 2 | 7 | .5 | 0 | 0 |
| 3 | 7 | .5 | ½ can | ½ can |
| 4 | 7 | .5 | full can | full can |
| 5 | 7 | .5 | full can | full can + 500 ug IV |
| | | | | Selenium |

\hat{ }“full can” is 500 mL of Intestamin ®

Following enrollment, Dipeptiven® and parenteral selenium was started as soon as possible and continued until death or discharge for a maximum of 21 days. Following initial resuscitation, Intestamin® began and was continued until nutrition support was discontinued, death, or discharge from ICU. Either study supplement could have been discontinued if an individual patient reached a pre-determined safety threshold. The final safety thresholds were determined after baseline data were collected (Group 1) and analyzed. If 3/7 patients in a group (42%) reach the threshold of safety, then no further dosing increments would occur but an additional 5 patients would be evaluated at the previous dosing range. All patients were fed according to clinical practice guidelines; enteral feeds will be initiated as per clinical practice.

When Dipeptiven® was provided, the daily dose was provided continuously via an intravenous central line over at least 20 of 24 hours. When Intestamin® was provided, the daily volume was infused via a nasogatric tube or nasoenteric feeding tube over at least 20 of 24 hours a day. Independent of glutamine/selenium dosing, all patients were fed according to clinical practice guidelines; enteral feeds were initiated and advanced as per clinical practice. When a patient was on both enteral feeds and Intestamin®, it required 2 pumps with the tubes attached directly to the two ports of a feeding tube or a “Y” connector was used to connect the tubing from the two pumps to one feeding tube in situ. With respect to both delays in administration of enteral feeds and the enteral study medications secondary to high gastric residuals, as is our common practice, motility agents and/or use of small bowel feeds were initiated with 24 hours of high gastric residual volumes or immediately in high risk patients (patients on continuous narcotics, inotropes, or paralytics and those patients who can not have the head of their bed elevated).

Outcomes: The primary outcome for this study is change (delta) sequential organ failure assessment (SOFA score). The secondary outcomes are glutathione, glutathione peroxidase, TBARS, IL-18, serum chemistries (BUN, AST, ALT, GGT, ammonia and plasma amino acid and dipeptide levels), tolerance of enteral nutrition, duration of mechanical ventilation, hospital length of stay, and 28 day mortality.

Multiple organ dysfunction is recognized as the final common pathway preceding death in critically ill patients. Each organ (respiratory, renal, etc.) can be considered in isolation or in the aggregate using scoring systems such as the Sequential Organ Failure Assessment (SOFA; see Table 6).

TABLE 6
Summary of SOFA scoring system.

| | SOFA score | | | |
|---|---|---|---|---|
| | 1 | 2 | 3 | 4 |
| Respiration | <400 | <300 | <200 | <100 |
| PaO₂/FiO₂ mmHg | | | with respiratory support | with respiratory support |
| Coagulation | <150 | <100 | <50 | <20 |
| Platelets × 10³/mm³ | | | | |
| Liver | 1.2-1.9 | 2.0-5.9 | 6.0-11.9 | >12.0 |
| Bilirubin, mg/dL | (20-32) | (33-101) | (102-204) | (>204) |
| (μmol/L) | | | | |
| Cardiovascular | MAP < 70 mmHg | Dopamine ≦ 5 or | Dopamine < 5 or | Dopamine > 1.5 or epinephrine > 0.1 |
| Hypotension^{a} | | Dobutamine (any dose) | epinephrine ≦ 0.1 or | or norepinephrine > 0.1 |
| | | | norepinephrine ≦ 0.1 | |
| Central Nervous System | 13-14 | 10-12 | 6-9 | <6 |
| Glasgow coma score | | | | |
| Renal | 1.2-1.9 | 2.0-3.4 | 3.5-4.9 | >5.0 |
| Creatinine, mg/dL | (110-170) | (171-299) | (300-440) or | (>440) or |
| (μmol/L) or urine output | | | <500 mL/day | <200 mL/day |

^{a}adrenergic agents administered for at least one hour (doses given are in μg/kg · min)

Upon enrollment (prior to initiation of study interventions) and daily thereafter while in ICU, daily parameters were measured that allow for calculation of the baseline, daily, total, and change in SOFA, for each organ system, and in the aggregate (white blood count, serum creatinine, arterial blood gases, fractional of inspired oxygen, blood pressure, use of vasopressors, urine output and Glasgow coma score). Based on what changes are observed in the control group, stopping rules were established, related to organ dysfunction, that if met, the study patient would have the study intervention withdrawn. On the basis of the control group the following stopping rule was established: with respect to the aggregate SOFA score a SOFA >3 increase from baseline for 2 or more days not attributable to underlying illness. The development of renal failure and/or the initiation of dialysis was not considered criteria for stopping, study interventions. All patients who have the study intervention withdrawn would, still be followed daily to evaluate their evolution of organ dysfunction (or resolution).

In addition to the above noted measurements, routine measurements of liver function tests (AST, ALT, GGT) and blood urea nitrogen were monitored when clinically available. From blood work drawn for routine clinical practice, a daily bilirubin and CRP were requested. In Groups 2, 3 and 4, 14 mls of blood from study patients were drawn at baseline, 12 mls of blood Monday, Wednesday, Friday while on the study protocol, and 12 hours following discontinuation of the Dipeptiven® and/or Intestamin®, and 2 mls of blood twice weekly (Tuesday and Thursday) while on the study protocol. This blood was processed, stored and sent to a laboratory for measurement of plasma ammonia, amino acid and dipeptide levels, and other markers including glutathione, glutathione peroxidase, and T-BARS. Finally, patients were followed to evaluate tolerance of enteral nutrition, duration of mechanical ventilation, hospital length of stay, and 28 day mortality.

Sample Size and Duration: 30 patients in prospective cohort which serves as a control group and 28 patients prospectively enrolled in dose-ranging studies from the KGH site over 6 months.

Significance: The therapeutic strategies tested in this dosing study illuminate desired dose and duration for glutamine and antioxidants. These results may be further used to inform a large, multicenter, Phase III randomized trial of glutamine and antioxidant supplementation in critically ill patients.

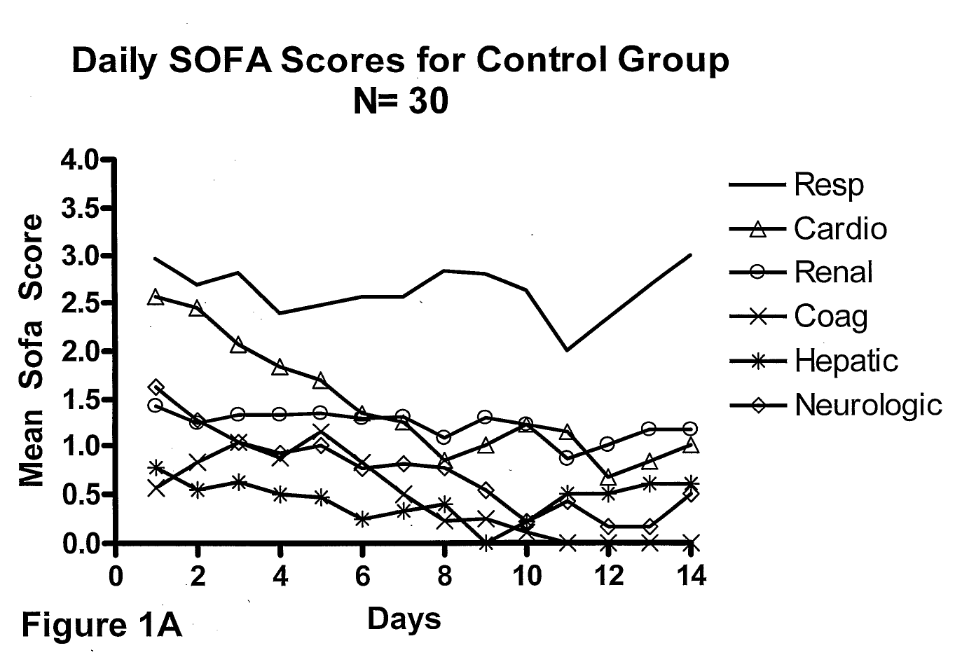

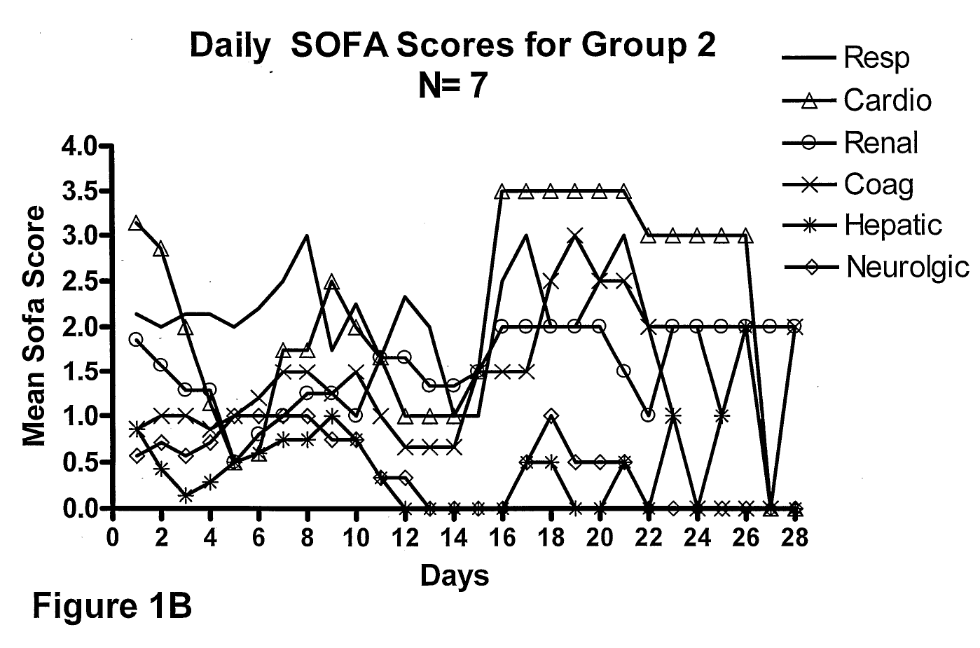

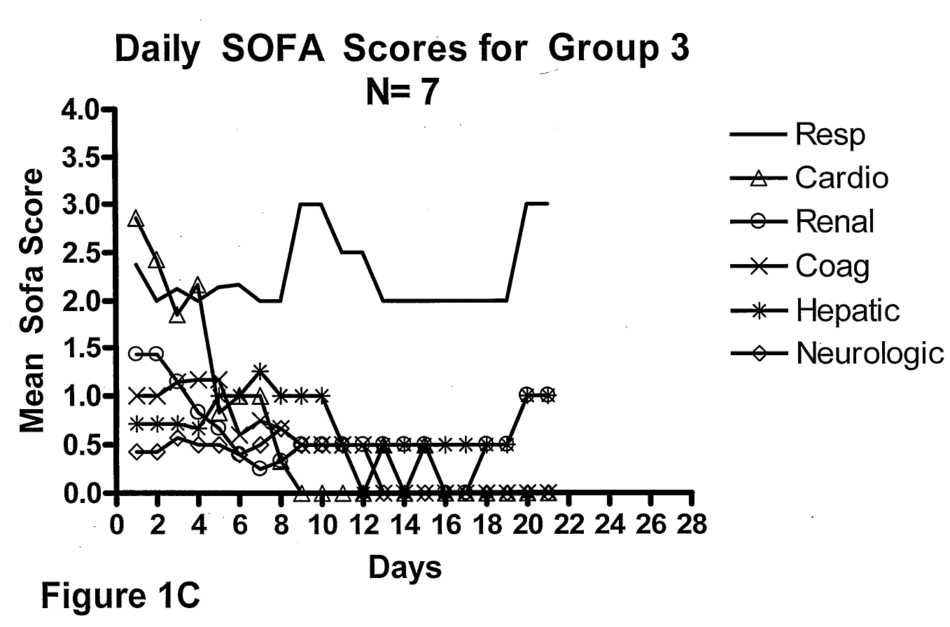

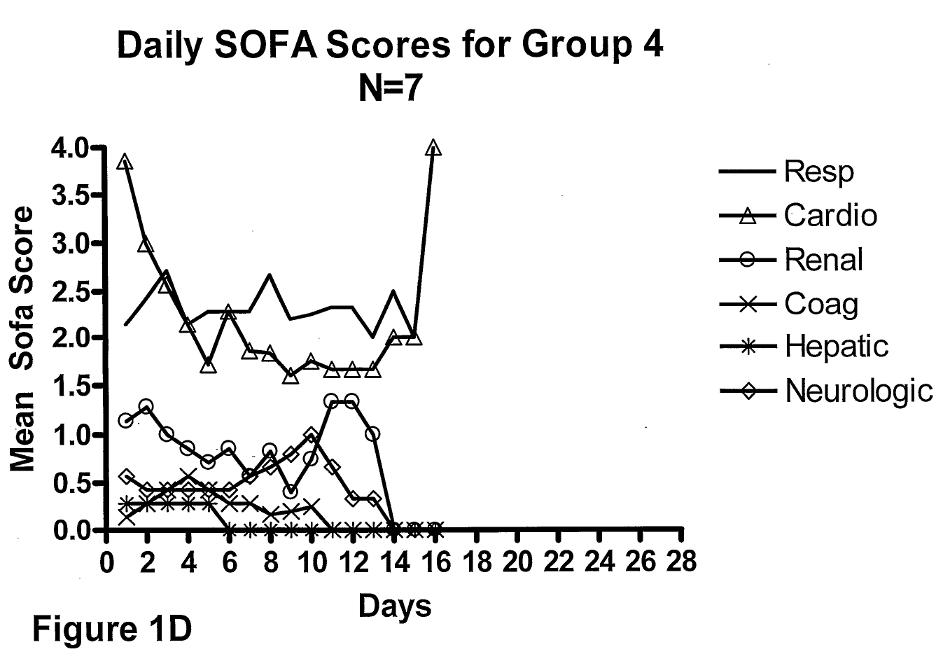

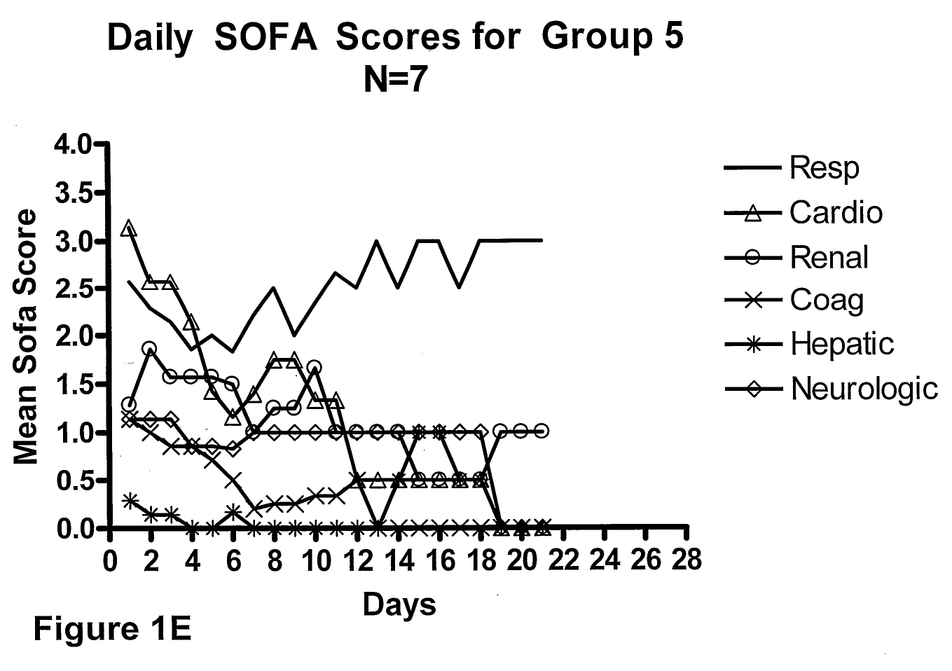

Results: 58 critically ill patients were enrolled over a two year period to receive escalating doses of glutamine and antioxidants (see Table 5 for summary of intervention). Daily SOFA scores for various organ systems (cardiovascular (CVS); central nervous system (CNS); coagulation; renal; liver; respiration (P/F ratio)) were determined for Groups 1 to 5. A; decrease in SOFA score indicates improvement. The mean daily SOFA scores for Groups 1 to 5 are shown in FIGS. 1A-1E, respectively.

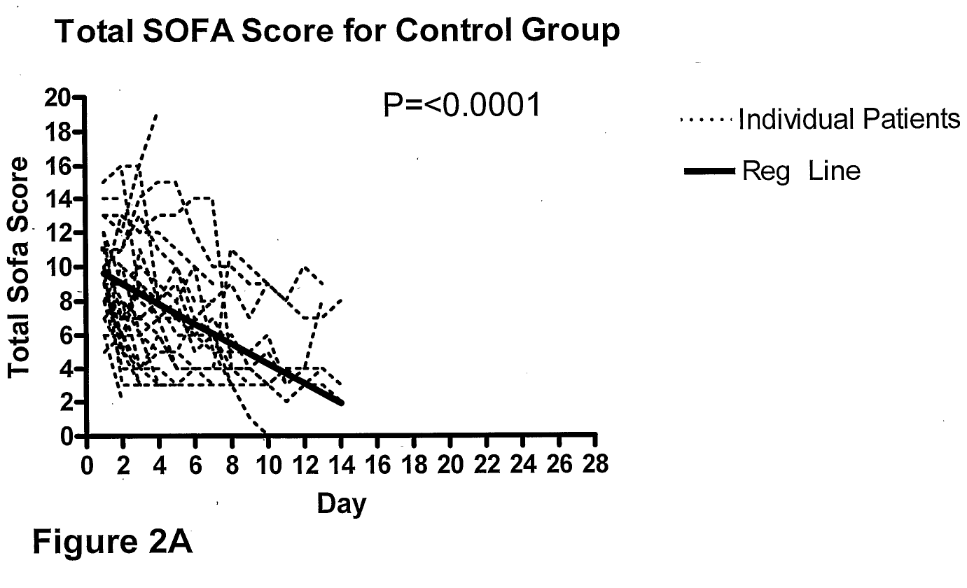

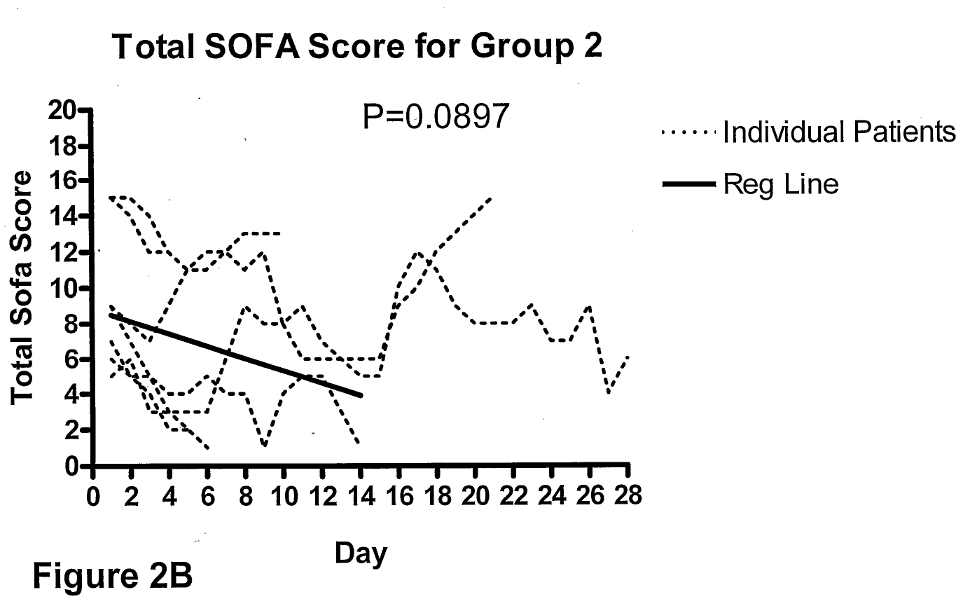

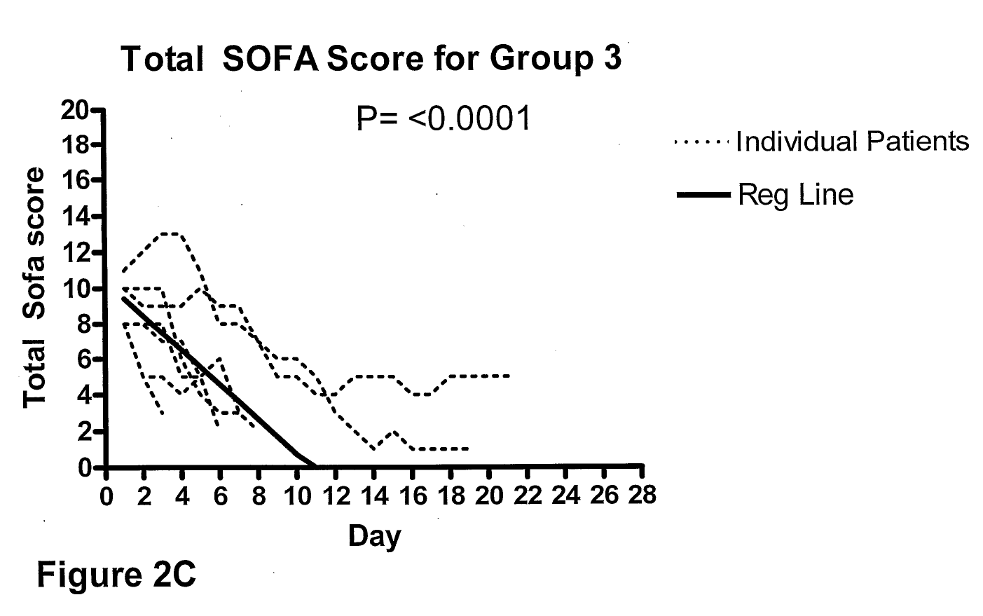

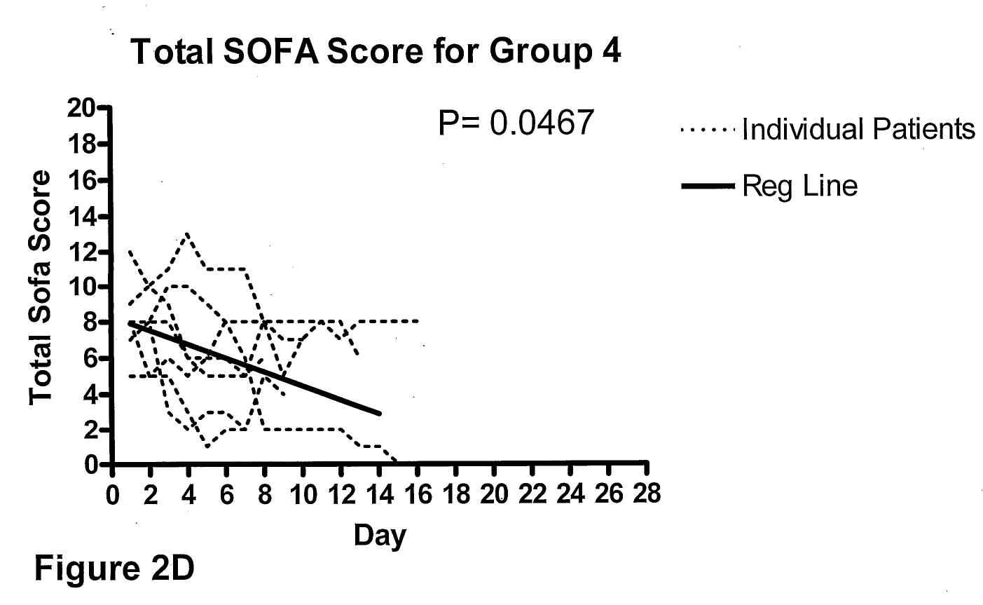

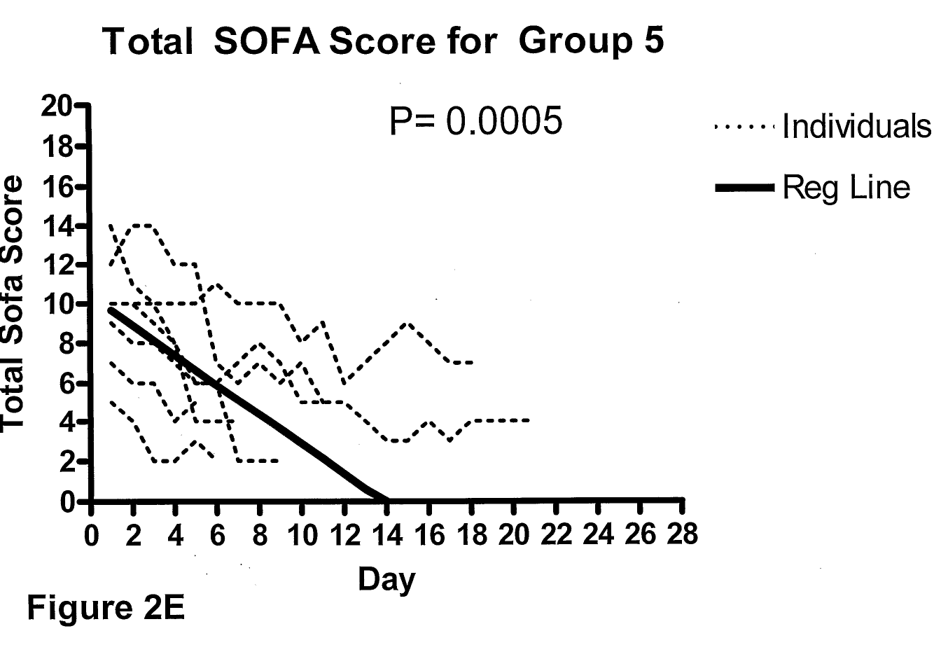

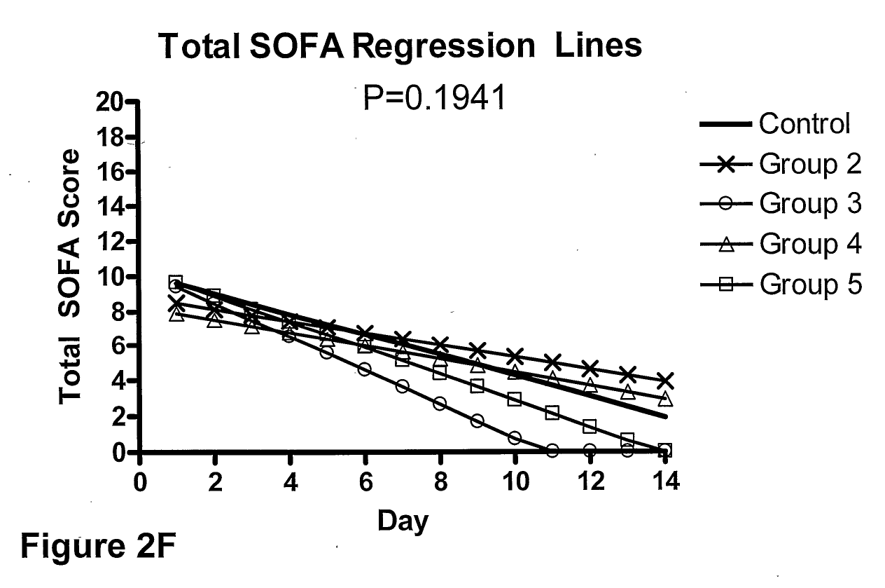

FIGS. 2A-2E show plots of total daily SOFA scores for individual patients in Groups 1 to 5, respectively. Regression lines compiled in FIG. 2F show that daily aggregate SOFA scores for groups 1 to 5 are similar and follow a similar decreasing trend throughout the study intervention indicating that the high doses of glutamine and antioxidants administered to Groups 2 to 5 were non-toxic and had no adverse effect on organ function. The increase in SOFA score shown in Group 2 (see range of day 6 to day 10 in FIG. 2B) were due to 2 out of 7 patients having a significant rise in their SOFA scores prior to dying. The deaths and increase in SOFA score of these two patients were found to be due to underlying disease and unrelated to the study intervention.

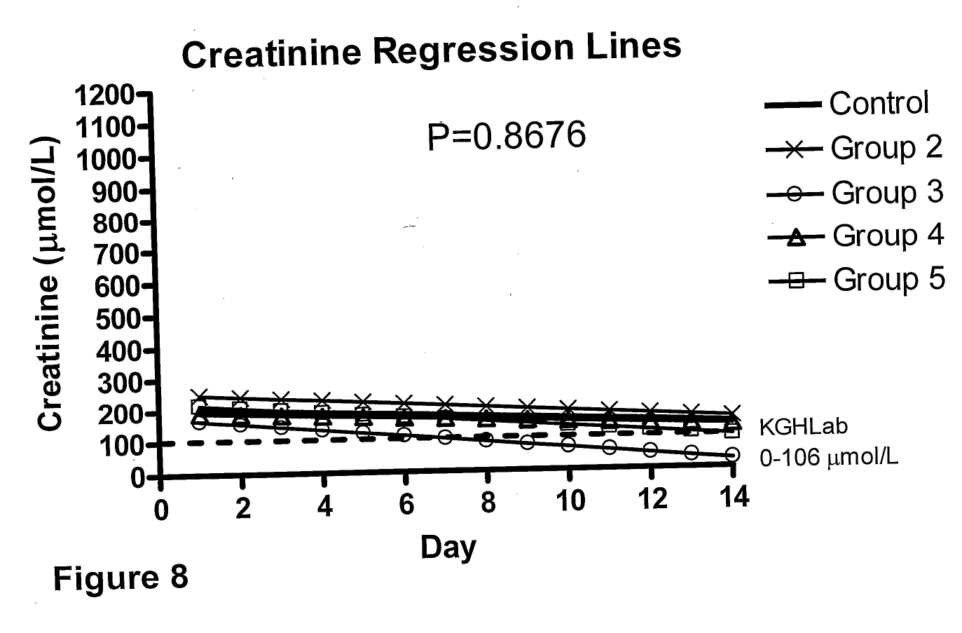

As glutamine is a nitrogen donor high doses of glutamine might be expected to increase urea and ammonia to undesirable levels. Determination of urea and ammonia levels showed a slight, but insignificant, increase. As would be expected, Selenium levels were significantly increased, particularly in Group 5. However, increased levels of these compounds did not adversely effect renal function as shown by stable creatinine levels in FIG. 8 and decreasing SOFA scores in FIGS. 1A-E.

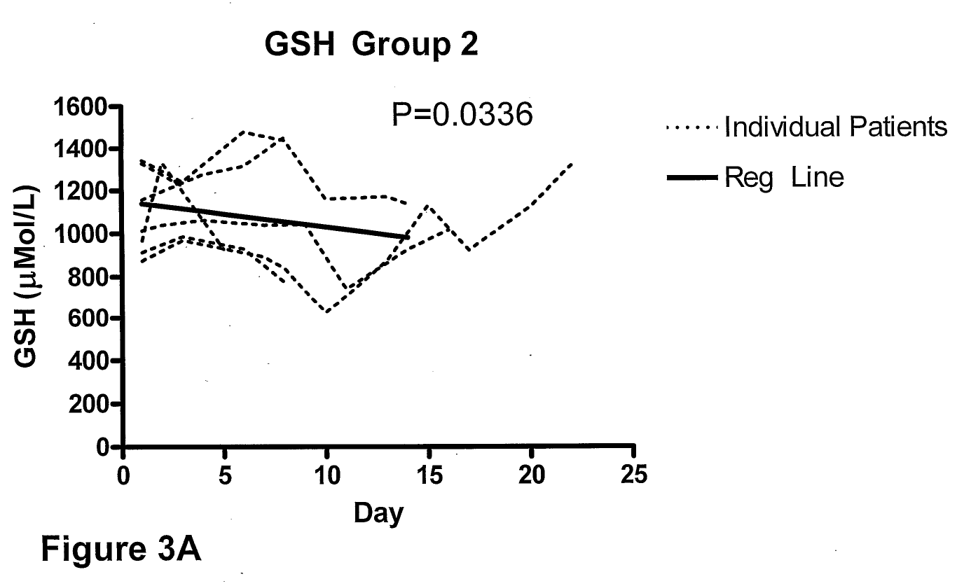

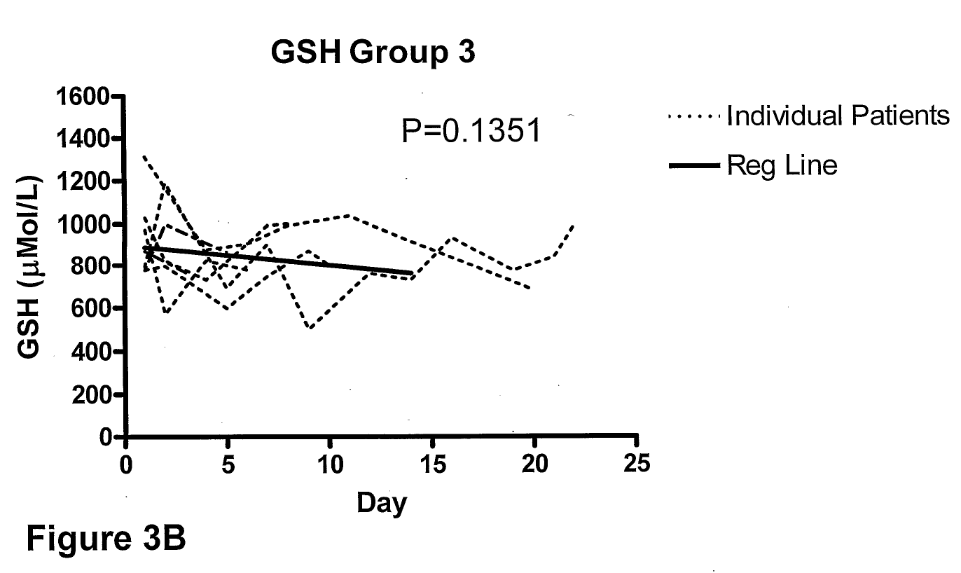

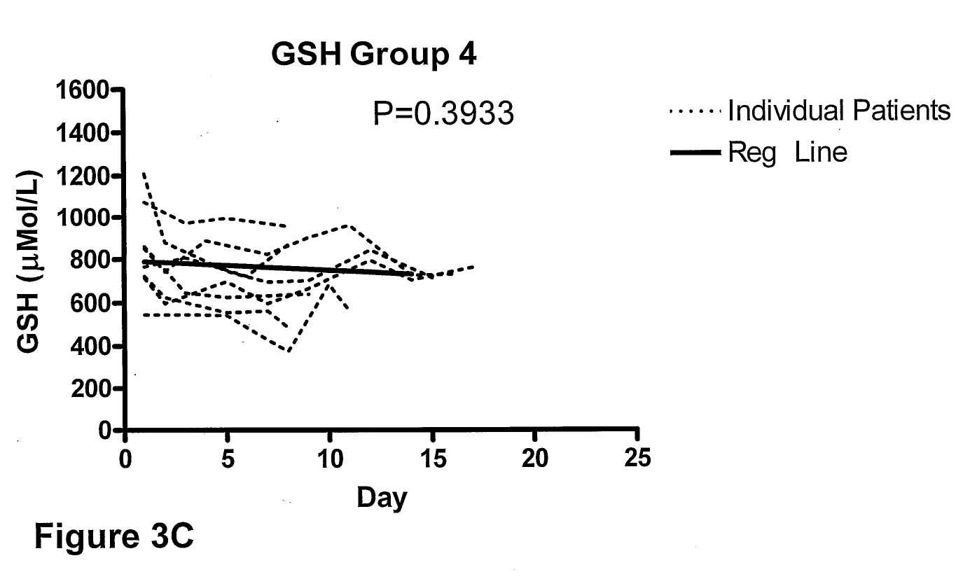

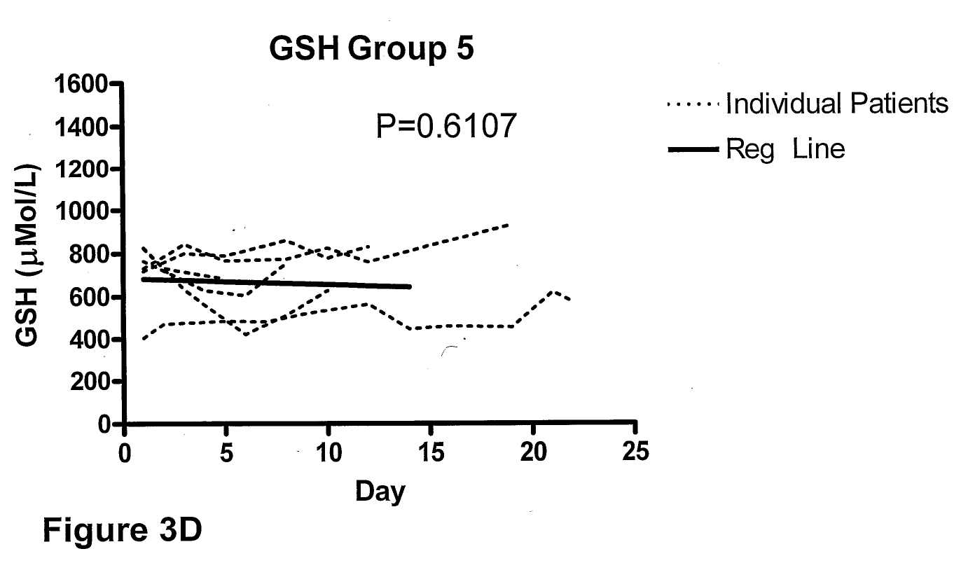

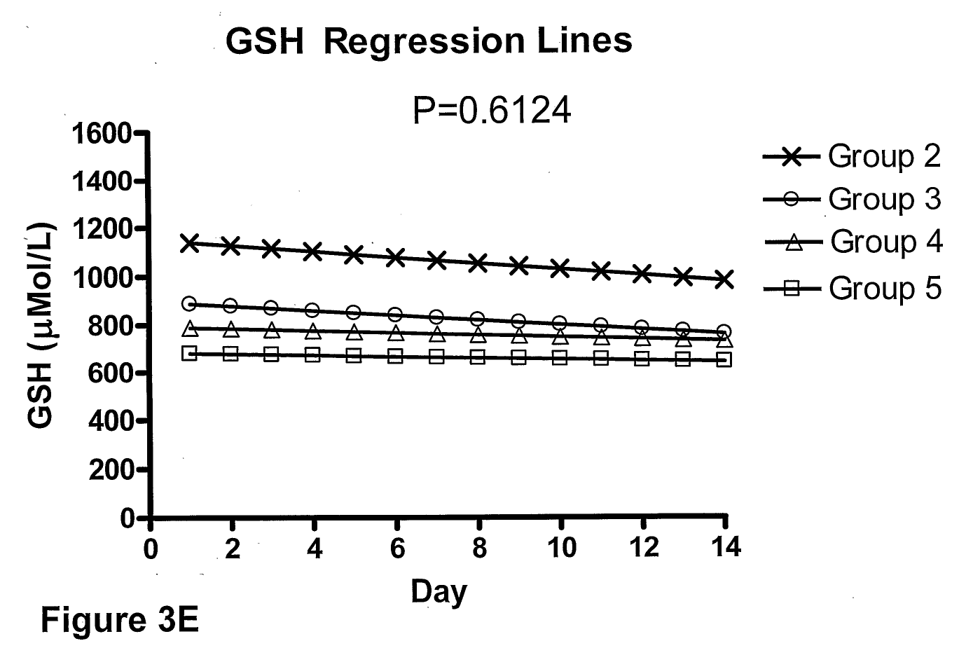

The effects of the study intervention on glutathione (GSH) content in red blood cells, markers of oxidative stress (TBARS), and index of mitochondrial function (mtDNA/nDNA) are shown in FIGS. 3 to 7.

FIG. 3 shows plots of glutathione (GSH) content of red blood cells for patients in Group 2 (FIG. 3A), Group 3 (FIG. 3B), Group 4 (FIG. 3C), and Group 5 (FIG. 3D) with the regression lines shown in a larger point size. The linear regression line for Group 2 demonstrates decreasing levels of GSH with a significant P value (P=0.0336). None of the other Groups 3 to 5 show this type of significant decrease. This result implies greater preservation of GSH levels in groups that received greater antioxidant supplementation.

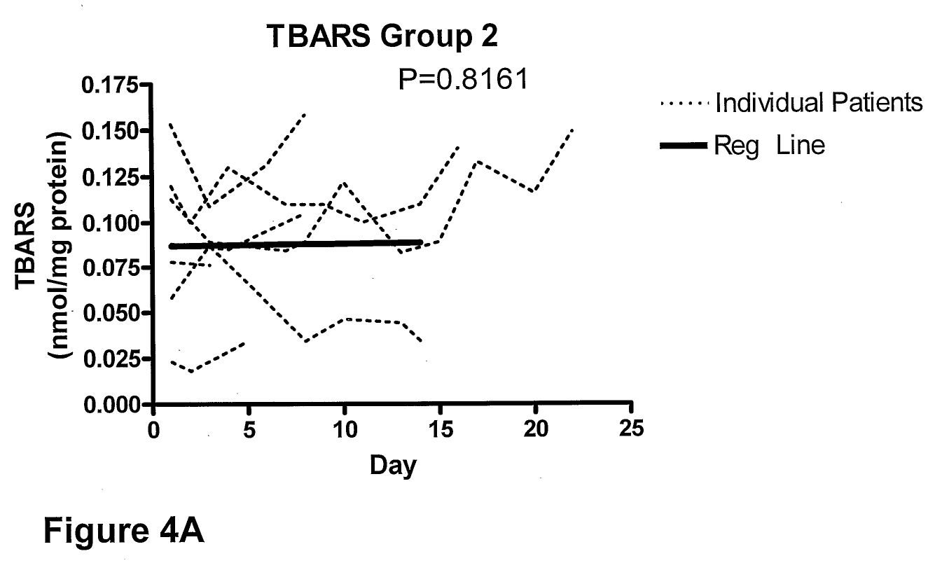

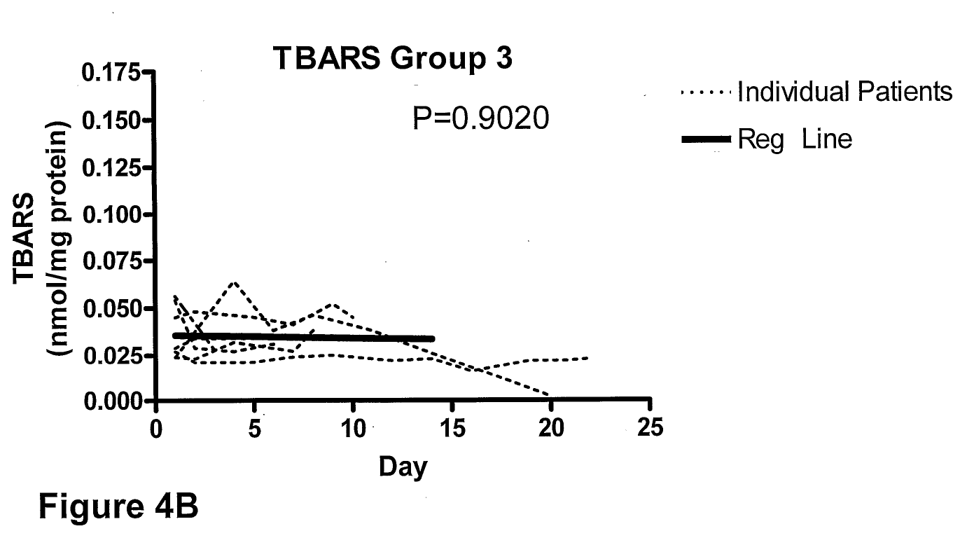

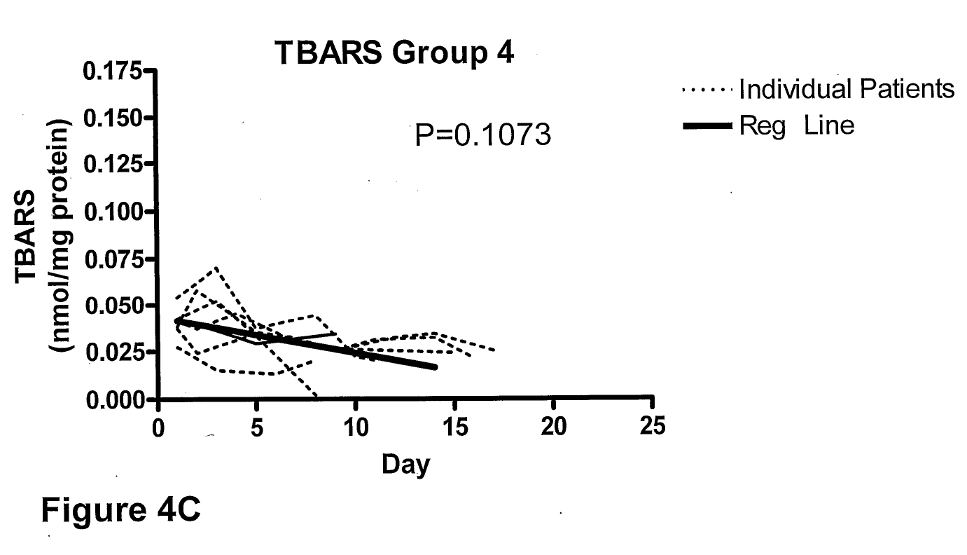

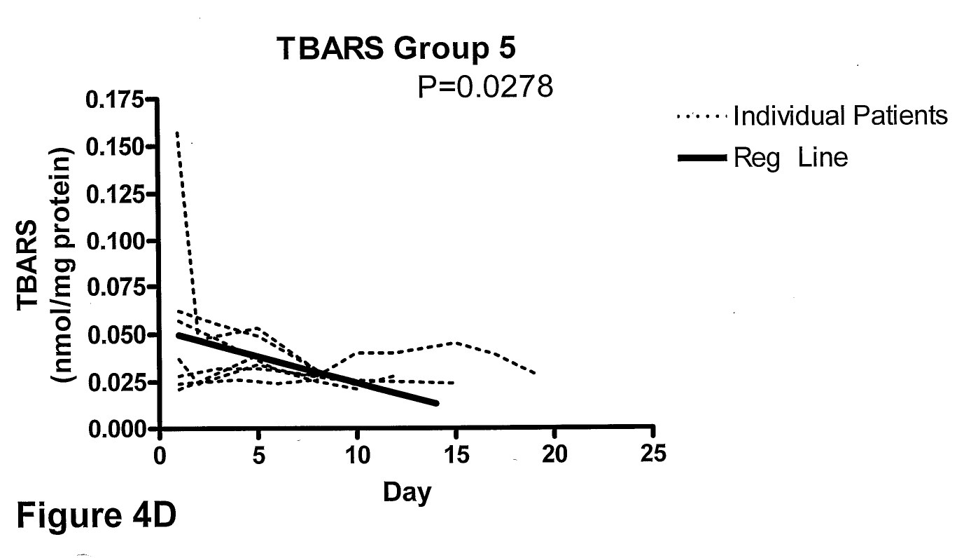

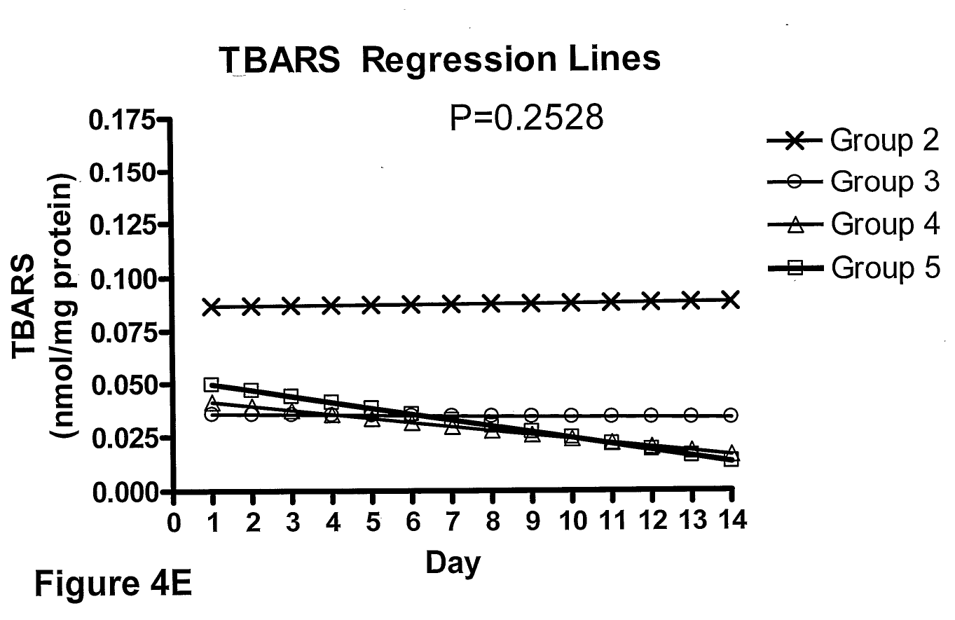

FIG. 4 shows plots of plasma concentrations of thiobarbituric acid reactive substances (TBARS), for patients in Group 2 (FIG. 4A), Group 3 (FIG. 4B), Group 4 (FIG. 4C), and Group 5 (FIG. 4D). TBARS analysis is used as a marker of oxidative stress. The linear regression lines for TBARS levels for Groups 2 to 4 do not achieve significant P values. However, the TBARS linear regression line for Group 5 shows a decreasing slope and does achieve significance (P=0.0278), implying that greater antioxidant supplementation may allow for improved resolution of oxidative stress.

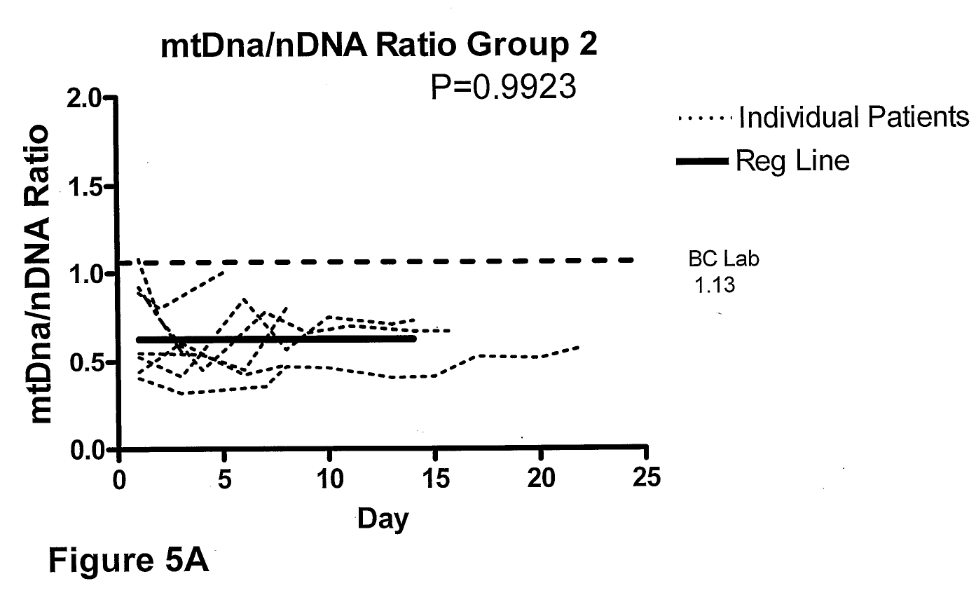

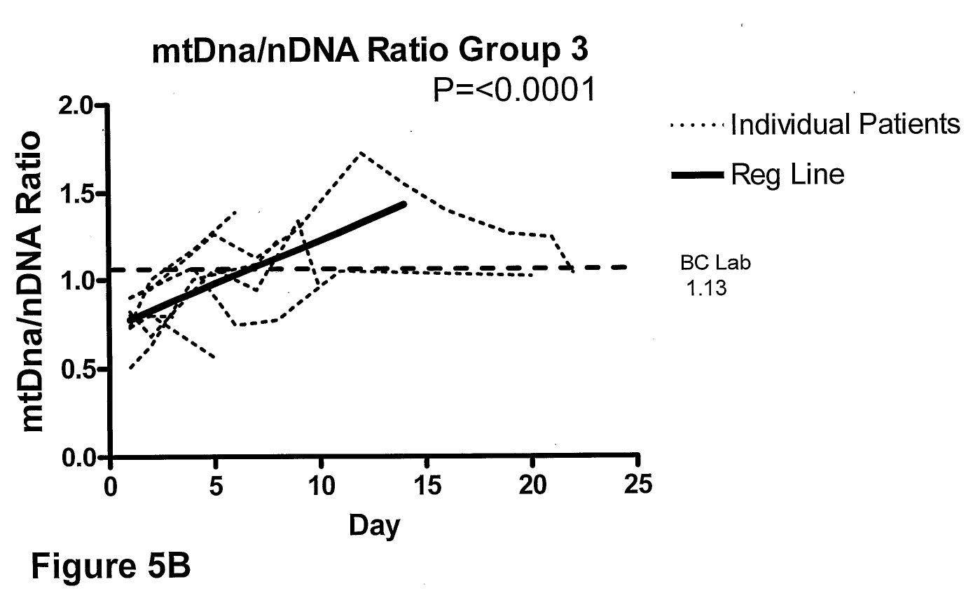

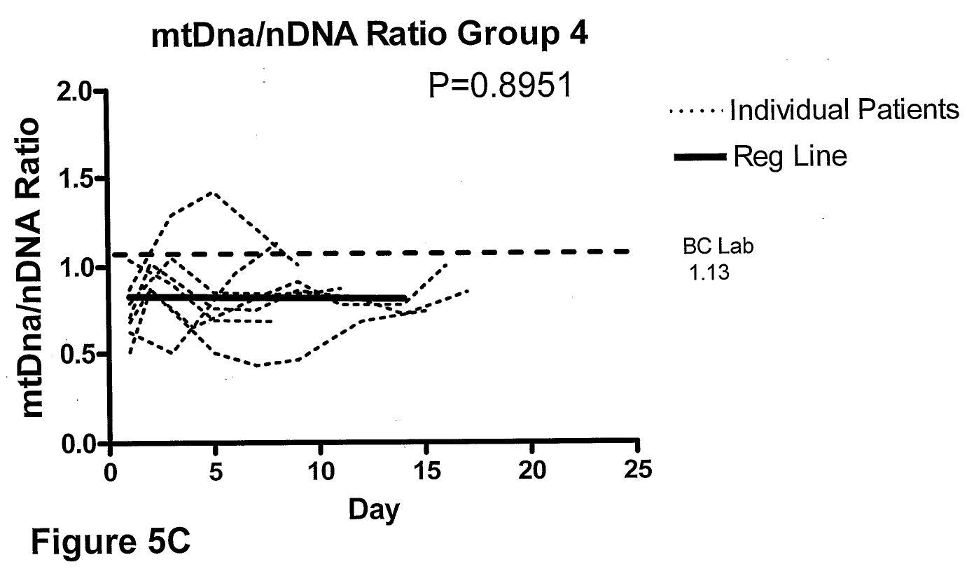

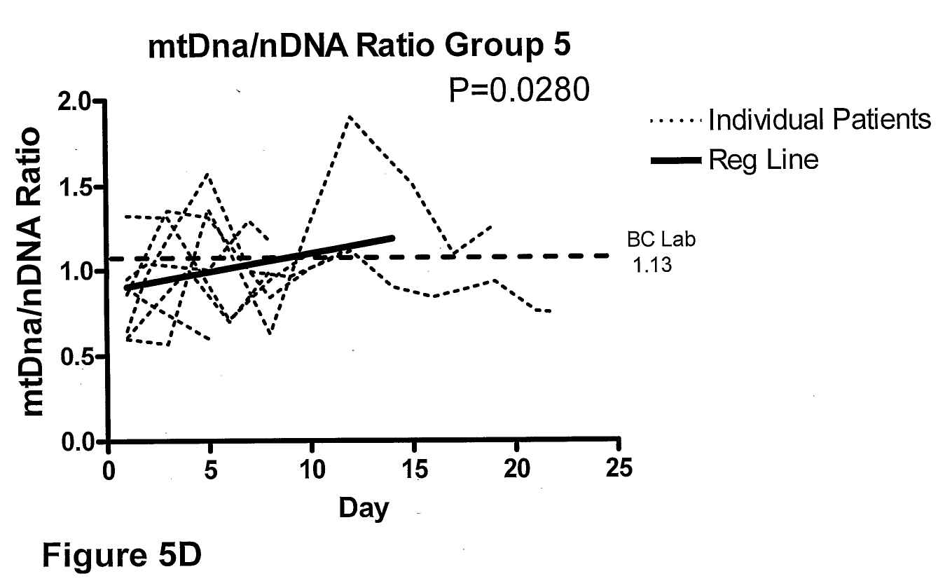

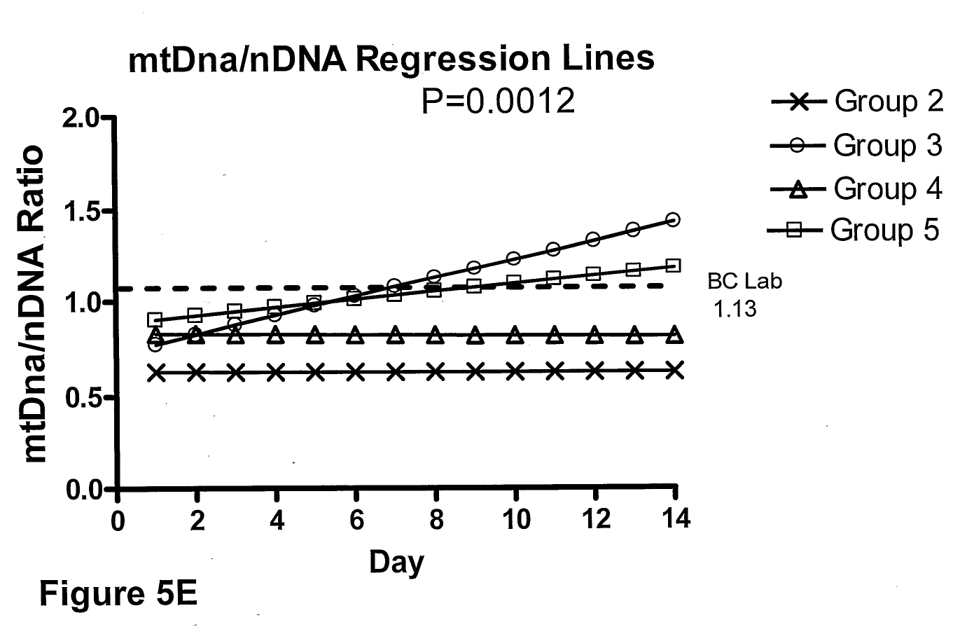

FIG. 5 shows plots of the ratio of levels of mitochondrial DNA and nuclear DNA (mtDNA/nDNA), for patients in Group 2 (FIG. 5A), Group 3 (FIG. 5B), Group 4 (FIG. 5C), and Group 5 (FIG. 5D). mtDNA/nDNA is an assay of mitochondrial function. The linear regression lines for Groups 3 and 5 show improved mitochondrial function during the course of treatment, and both regression lines show significant P values (P<0.0001 and P=0.0280, respectively). Furthermore, compilation of linear regression lines for Groups 2 to 5 on a single plot (FIG. 5E) also achieves significance (P=0.0012).

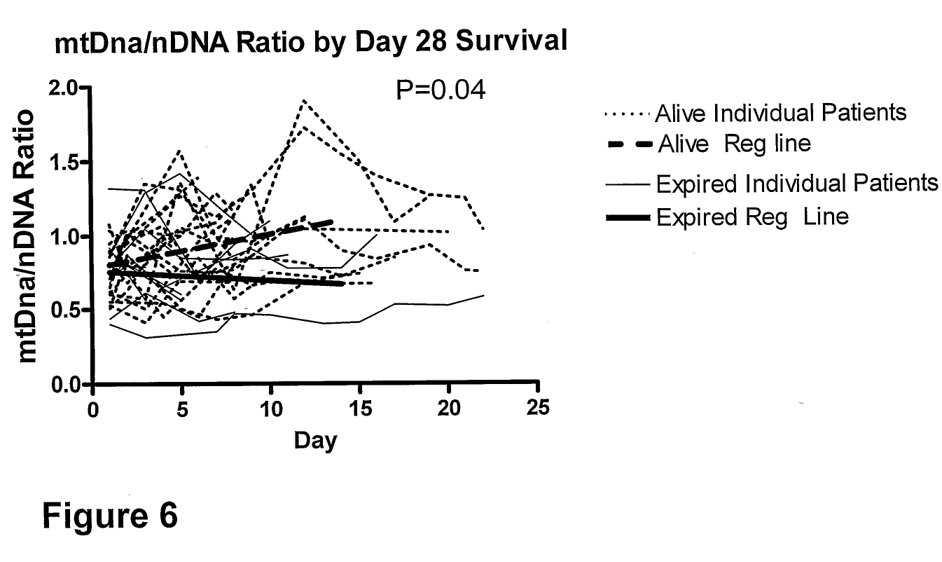

FIG. 6 shows plots of the mtDNA/nDNA ratio for individual patients that are categorized as either “alive” or “expired” with regression lines shown in a larger point size. This result shows that improved mitochondrial function is clearly correlated with survival, as the linear regression lines for “alive” and “expired” patients achieve significance (P=0.04).

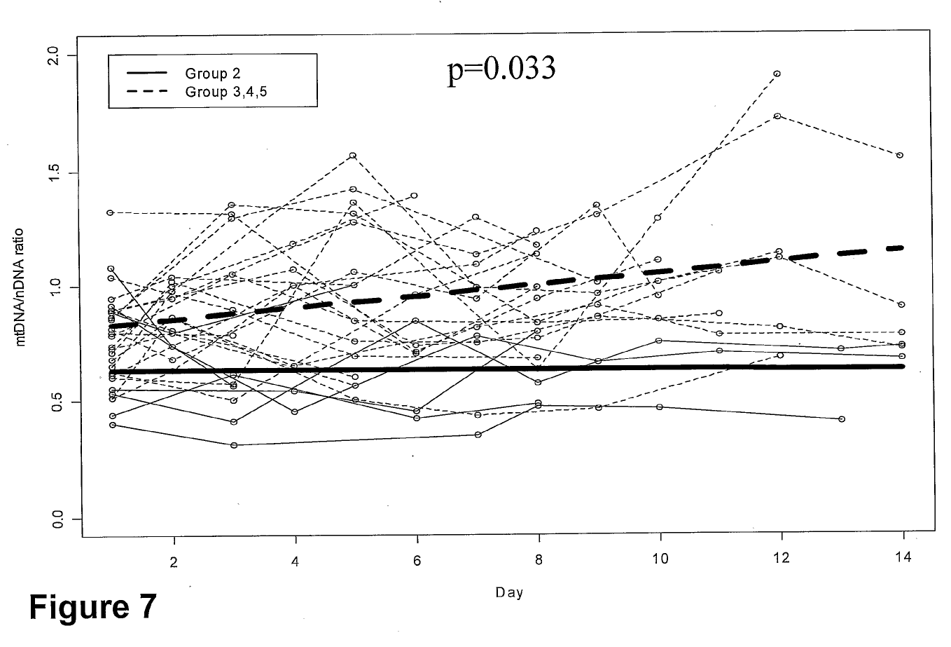

FIG. 7 shows plots of the mtDNA/nDNA ratio for individual patients that are categorized as either Group 2 patients or Groups 3, 4, and 5 patients with regression lines shown in a larger point size. Again, the linear regression lines achieve significance (P=0.033). This result shows that each of Groups 3, 4, and 5 demonstrate significant improvement in mitochondrial function in comparison to Group 2, suggesting that antioxidant and glutamine supplementation can improve mitochondrial function.

The data in these Figures indicate that the effects of escalating the dose of glutamine and antioxidants is improved mitochondrial function, a greater reduction in markers of oxidative stress, greater preservation of glutathione, with no apparent adverse effect on organ function. Furthermore, there was no worsening of inflammatory cytokines as exemplified by stable levels of IL-18 (data not shown).

All citations are hereby incorporated by reference.

The present invention has been described with regard to one or more embodiments. However, it will be apparent to persons skilled in the art that a number of variations and modifications can be made without departing from the scope of the invention as defined in the claims.

1. A composition comprising glutamine from about 35 to about 380 grams per litre of solution provided as a short chain peptide, and an antioxidant selected from the group consisting of selenium at a concentration from about 400 to about 10000 micrograms per litre, vitamin C at a concentration from about 1000 to about 20000 milligrams per litre, zinc at a concentration from about 20 to about 800 milligrams per litre, vitamin E at a concentration from about 500 to about 12000 milligrams per litre, and beta [symbol] carotene at a concentration from about 20 to about 4000 milligrams per litre.

2. The composition according to claim 1, wherein said antioxidant is selenium at a concentration from about 1000 to about 4000 micrograms per litre.

3. The composition according to claim 1, wherein said concentration of glutamine is from about 50 to about 150 grams per litre of solution.

4. The composition according to claim 2, wherein lipids or carbohydrates are absent.

5. A unit dosage form of about 50 to about 1000 millilitres total volume containing said composition of claim 1.

6. The unit dosage form according to claim 5, wherein, said antioxidant is selenium at a concentration from about 1000 to about 4000 micrograms per litre.

7. The unit dosage form according to claim 5, wherein said concentration of glutamine is from about 50 to about 150 grams per litre of solution.

8. The, unit dosage form according to claim 5, wherein said total volume is about 50 to about 500 millilitres.

9. A method of treating a critically ill patient comprising parenterally administering said composition of claim 1, to a critically ill patient in need of such treatment.

10. The method according to claim 9, wherein said composition is administered to said patient in a daily dose from about 0.3 g glutamine/kg bodyweight to about 0.9 g glutamine/kg body weight.

11. The method according to claim 9, wherein said antioxidant is selenium and said composition is administered to said patient in a daily dose from about 400 to about 2000 micrograms.

12. A method of treating a critically ill patient comprising parenterally administering said composition of claim 4, to a critically ill patient in need of such treatment.

13. The method according to claim 12, wherein said composition is administered to said patient in a daily dose from about 0.3 g glutamine/kg body weight to about 0.9 g glutamine/kg body weight.

14. The method according to claim 12, wherein said antioxidant is selenium and said composition is administered to said patient in a daily dose from about 400 to about 2000 micrograms.

15. A method for improving mitochondrial function comprising parenterally administering said composition of claim 1, to a patient in need of such treatment.

16. The method according to claim 15, wherein said composition is administered to said patient in a daily dose from about 0.3 g glutamine/kg body weight to about 0.9 g glutamine/kg body weight.

17. The method according to claim 15, wherein said antioxidant is selenium and said composition is administered to said patient in a daily dose from about 400 to about 2000 micrograms.

18. A method for improving mitochondrial function comprising parenterally administering said composition of claim 4, to a patient in need of such treatment.

19. The method according to claim 18, wherein said composition is administered to said patient in a daily dose from about 0.3 g glutamine/kg body weight to about 0.9 g glutamine/kg body weight.

20. The method according to claim 18, wherein said antioxidant is selenium and said composition is administered to said patient in a daily dose from about 400 to about 2000 micrograms.

21. The composition according to claim 1, wherein said composition is to be delivered parenterally to a critically ill patient.

22. The composition according to claim 4, wherein said composition is to be delivered parenterally to a critically ill patient.

23. The composition according to claim 1, wherein said composition is to be delivered parenterally to improve mitochondrial function in a patient.

24. The composition according to claim 4, wherein said composition is to be delivered parenterally to improve mitochondrial function in a patient.

25. The unit dosage form according to claim 5, wherein said composition is to be delivered parenterally to a critically ill patient.

26. The unit dosage form according to claim 5, wherein said composition is to be delivered parenterally to improve mitochondrial function in a patient.

27. A combination comprising glutamine from about 35 to about 380 grams per litre of solution provided as a short chain peptide, and an antioxidant selected from the group consisting of selenium at a concentration from about 400 to about 10000 micrograms per litre, vitamin C at a concentration from about 1000 to about 20000 milligrams per litre, zinc at a concentration from about 20 to about 800 milligrams per litre, vitamin E at a concentration from about 500 to about 12000 milligrams per litre, and beta carotene at a concentration from about 20 to about 4000 milligrams per litre.

28. The combination according to claim 27, wherein said antioxidant is selenium at a concentration from about 1000 to about 4000 micrograms per litre.

29. The combination according to claim 27, wherein said concentration of glutamine is from about 50 to about 150 grams per litre of solution.

30. The combination according to claim 28, wherein lipids or carbohydrates are absent.

31. A method of treating a critically ill patient comprising parenterally administering said combination of claim 27, to a critically ill patient in need of such treatment.

32. A method of treating a critically ill patient comprising parenterally administering said combination of claim 30, to a critically ill patient in need of such treatment.

33. A method for improving mitochondrial function comprising parenterally administering said combination of claim 27, to a patient in need of such treatment.

34. A method for improving mitochondrial function comprising parenterally administering said combination of claim 30, to a patient in need of such treatment.

35. The method of claim 31, wherein said glutamine and said antioxidant are delivered simultaneously.

36. The method of claim 31, wherein said glutamine and said antioxidant are delivered separately.
